(19) United States
(12) Patent Application Publication
(10) Pub. No.: US 2015/0018294 A1
(43) Pub. Date: Jan. 15, 2015

(54) MODULATORS OF TOUSLED KINASE IN CELLULAR PROCESSES

(71) Applicants: Arrigo DE BENEDETTI, Shreveport, LA (US); Gulshan SUNAVALA, Shreveport, LA (US)

(72) Inventors: Arrigo DeBenedetti, Shreveport, LA (US); Gulshan Sunavala, Shreveport, LA (US)

(21) Appl. No.: 14/377,457

(22) PCT Filed: Feb. 7, 2013
(86) PCT No.: PCT/US13/25165

§ 371 Date: Aug. 7, 2014

Related U.S. Application Data
(60) Provisional application No. 61/595,781, filed on Feb. 7, 2012.

(51) Int. Cl. A61K 31/5415; A61N 5/10; A61K 31/7064; A61K 31/553; A61K 31/65; A61K 31/22

(52) U.S. Cl. CPC A61K 31/5415; A61K 31/65; A61K 31/22; A61K 31/7064; A61K 31/553; A61N 5/1001; A61N 2005/1098
USPC 514/32; 514/225.8; 514/154; 514/546; 514/211.08; 514/226.2

(57) ABSTRACT

The present invention relates to compounds useful as inhibitors of protein kinase. The invention also provides pharmaceutically acceptable compositions comprising said compounds and methods of using the compositions in the treatment of various disease, conditions, or disorders. The invention also provides for methods and pharmaceutical agents to modulate the activity of Tousled-like kinase (TLK). The invention also provides for methods and pharmaceutical agents to inhibit the activity of Tousled-like kinase to provide increased sensitivity to irradiation (IR) and chemotherapeutic agents. The invention also provides for methods and pharmaceutical agents to increase the activity of Tousled-like kinase to provide increased protection against DNA damaging agents including to irradiation (IR) and chemotherapeutic agents.

### CROSS REFERENCE TO RELATED APPLICATIONS

This application claims the benefit of U.S. Provisional Patent Application 61/595,781, entitled “MODULATORS OF TOUSLED KINASE IN CELLULAR PROCESSES” filed Feb. 7, 2012, the entire contents of which are incorporated herein by reference.

### STATEMENT REGARDING FEDERALLY SPONSORED RESEARCH OR DEVELOPMENT

This research was made possible by funding under grant number W81XWH-10-1-0120 IDEA Development Award awarded by the Department of Defense. The government may have certain rights in this invention.

### BACKGROUND OF THE INVENTION

1. Field of the Invention

The present invention relates to compounds useful as inhibitors of protein kinase. The invention also provides pharmaceutically acceptable compositions comprising said compounds and methods of using the compositions in the treatment of various disease, conditions, or disorders. The invention also provides for methods and pharmaceutical agents to modulate the activity of Tousled-like kinase (TLK). The invention also provides for methods and pharmaceutical agents to inhibit the activity of Tousled-like kinase to provide increased sensitivity to irradiation (IR) and chemotherapeutic agents. The invention also provides for methods and pharmaceutical agents to increase the activity of Tousled-like kinase to provide increased protection against DNA damaging agents including to irradiation (IR) and chemotherapeutic agents.

2. Description of the Related Art

Much work in cancer therapy is devoted to the problem of drug resistance. However, resistance is a late outcome that could be avoided by the implementation of better combination therapies to reduce the RMT doses while still specifically targeting cancer cells.

Prostate cancer (CaP) is newly diagnosed in over 200,000 men each year and results in around 40,000 annual disease-related deaths in the US. Organ-confined prostate cancer is generally treated by surgical removal or irradiation of the whole gland, but nearly 30% of those undergoing either surgery or radiation will have recurrent disease at local (radiation therapy) or distant (radiation or surgery) sites in less than 7 Years.

Radical prostatectomy (RP) and radiation therapy (RT) are the primary treatment options for organ-confined prostate cancer (T1-T2, stages I or II). Eventually, about 50% to 70% of post prostatectomy patients with high-risk pathologic features such as a positive margin, extra-capsular extension (HCE), or seminal vesicle involvement (SVI) will develop biochemical failure (BF). Therefore, RT may play a role either immediately following prostatectomy (based on various known high-risk pathologic features) or at the time of BF.

Success for radiation therapy (XRT) and/or radiomimetic therapy (RMT) for CaP is based on the assumption that dividing cancer cells are preferentially killed with respect to non-dividing normal tissues. There are two major problems with XRT-based approaches to CaP: 1) For some patients, their cancer cells may be inherently resistant to XRT (often because of efficient DNA repair) resulting in residual resistant cancer cells at the primary site (local recurrence), and 2) intestinal mucosa adjacent to the prostate contains rapidly dividing, self-renewing cells that are highly susceptible to XRT, resulting in the most significant side effect limiting its utility because of high toxicity. Therefore, being able to identify compounds that can sensitize refractory cancers with lower doses of radiation and/or radiomimetic drug would constitute a major step forward in the use of XRT for the definitive treatment of organ-confined CaP. This would also aid treatment of advanced disease as cells seeding distant sites would not be inherently resistant to DNA damaging agents.

The tousled like kinases (TLKs) are involved in chromatin assembly, DNA repair, and transcription. Two TLK genes exist in humans and their expression is often dysregulated in cancer. TLKs phosphorylate Asf1 and Rad9, regulating Double-Strand Break (DSB) repair and the DNA Damage Response (DDR). TLKs maintain genomic stability and are important therapeutic intervention targets.

The Tousled locus was originally identified in A. Thaliana and Antirrhinum majus during a study of mutations leading to defects in meristem expansion. Mutations of Tousled produce a complex phenotype characterized by specific defects in development of leaf and floral organs. This was proposed to be linked to a replicative defect during organogenesis, but which may also result from failure to protect the genome from DNA damage, resulting in developmental aberrations.

Highly related Tousled-like genes can be found in many organisms in both kingdoms, several of which encode multiple transcripts resulting in different protein isoforms. It was originally proposed that Tousled (TSL) may be a component in a signal transduction pathway controlling cell proliferation and DNA synthesis during organogenesis, and this immediately prompted a search for its substrates. However, unlike most kinases that usually display a propensity to phosphorylate numerous substrates, after many years of study, only a few direct “interacting” substrates of TLKs have been identified, namely the histone chaperone Asf1, histone H3-S10, Aurora B, and more recently Rad9. This suggested a function for TLKs in chromatin assembly, during transcription; DNA repair; condensation of chromosomes at mitosis. The latter function, which was found critical for proper chromosome segregation, prompted a search for additional “indirect” substrates and functions, and resulted in the identification of an activity on myosin II in mammalian cells and on the chromosome passenger complex in trypanosomes.

A high percentage of human tumors, including cancer of the prostate (CaP) and breast (BCA), show mutations in DNA repair genes and checkpoint functions that make them overly dependent on alternative pathways for survival. Unfortunately, this can result in carcinomas that are highly resistant to radiation therapy (XRT) or radiomimetic therapy (RMT) from failsafe repair mechanisms also designed to contain excessive genomic instability. Targeting those mechanisms can result in highly specific and effective therapies. Clearly, some success from the adjuvant use of PARP inhibitors to treat BRCA mutants and/or triple negative BCA1 and other strategies to exploit DNA repair defects point in that direction. We propose that the addition of newly identified inhibitors of Tousled like kinases (TLKs) to enhance the response to XRT/RMT will greatly benefit the management of CaP and BCA patients. In fact, ameliorating the adverse effects of standard therapy, by reducing the doses while maintaining specific cancer killing, still seems to be the one of most promising courses of action for the near future. The TLKs are involved in chromatin assembly, DNA repair, transcription, and chromosome segregation. Two TLK genes (TLK1 and TLK2) with several splice variants were identified in humans. TLK1/1B interacts specifically with the chromatin assembly factor Asf1 and Rad9, and we have presented evidence that TLK1B promotes repair by processing the DSB ends and disassembling chromatin nearby to facilitate recruitment of repair proteins. Since Rad9 is a critical mediator of the DNA Damage Response (DDR), and in repair (specifically of DSBs), it seemed that the TLK1 Rad9 interaction would be very important in implementing TLK1B-mediated radioprotection. The past few years have witnessed significant advances in understanding the roles of TLKs in the DDR and in DSB repair, as well as their clinical relevance. In BCA, elevated expression of the TLK1B splice form is found in ˜30% of the patients and often corresponds to poor response to XRT and doxorubicin (doxo), presumably due to efficient DSB repair in the tumor cells.

In addition, there are BCA cases where TLK1/1B is not elevated, but TLK2 is amplified and/or overexpressed. Thus, for a large proportion of sporadic BCA, specific TLK inhibitors should be extremely beneficial as radio and chemo sensitizers. The fact that TLKs are overexpressed likely renders tumor cells more dependent on these kinases than normal tissues and hence, their preferential TLK targeted killing. In contrast to BCA, in the most common human CaP cell lines, only one or the other TLK gene is expressed, although typically at high levels 11—we do not have the story yet for the analysis of patient samples. If this represents a typical situation for CaP, then such cells would be even more dependent on the expression of either TLK, and hence their preferential killing with an inhibitor. In this work, we report the identification of some specific inhibitors of TLK, and surprisingly, some of these belong to the family of phenothiazine antipsychotics that have been approved for the treatment of schizophrenia for years.

The present invention has now identified two interacting proteins, Rad9 and TLK1 B, as critical mediators of XRT-refractory cancers and determined a novel mechanism of TLK1 B-mediated radio-resistance through modulation of Rad9 binding to double strand breaks (DSBs). The identification of TLK inhibitors offers the promise to approach XRT as the treatment of choice for first-line CaP cure or as adjuvant therapy post-surgery, as they will be extremely helpful as radio- or chemo-sensitizers. From a biochemical screen conducted in the Feist-Weiller Cancer Center's Small Molecule Screening Core Facility, we identified inhibitors of TLK1B that were primarily in a class of drugs presently used clinically as antipsychotic drugs.

### SUMMARY OF THE INVENTION

This invention relates to compositions and methods useful for treating various cancers. Therapeutic combinations and methods of use thereof are also covered in the present application.

The present invention relates to a pharmaceutical combination for the treatment of diseases, which involves cell proliferation, migration or apoptosis of myeloma cells, or angiogenesis. The invention also relates to a method for the treatment of said diseases, comprising co-administration of effective amounts of specific active compounds and/or co-treatment with radiation therapy, in a ratio which provides an additive and synergistic effect, and to the combined use of these specific compounds and/or radiotherapy for the manufacture of corresponding pharmaceutical combination preparations.

In one aspect, the present disclosure provides for methods and compositions to inhibit DSB repair and potentiate tumor cells killing with radiation therapy and/or radiomimetic therapy. In another embodiment, the present invention provides for anti-tumor therapy.

It has now been found that co-administration to a person in need of such treatment and/or co-treatment of a person in need of such treatment with effective amounts of (i) a selected tousled-like kinase inhibitor, and (ii) at least a further chemotherapeutic or naturally occurring, semi-synthetic or synthetic therapeutic agent, and/or radiotherapy or radioimmunotherapy, provides unexpected advantages in the treatment of diseases in which cell proliferation, migration or apoptosis of myeloma cells, or angiogenesis are involved, to a person in need of such treatment, with high efficacy, in comparison to administration of any of these substances alone and/or treatment with radiotherapy or radioimmunotherapy.

It has been further found that this co-administration or co-treatment is especially efficient if the selected tousled-like kinase inhibitor is an inhibitor of TLK1. In another embodiment, the TLK is TLK1B.

Further it has been found that the combination treatment in accordance with the present invention is especially efficient for inhibiting tumor growth, survival and metastasis.

Further it has been found that the combination treatment in accordance with the present invention is especially efficient with selected active substances, selected dosages and selected dosage forms.

Thus, the present invention provides a method for the treatment of diseases involving cell proliferation, migration or apoptosis of myeloma cells, or angiogenesis, which method comprises simultaneous, separate or sequential co-administration of effective amounts of: (i) an inhibitor of TLK or optionally in form of its tautomers, racemates, enantiomers, diastereomers and the mixtures thereof and optionally in form of the pharmacologically acceptable acid addition salts, solvates, hydrates, polymorphs, physiologically functional derivatives or prodrugs thereof; and (ii) at least a further chemotherapeutic or naturally occurring, semi-synthetic or synthetic therapeutic agent; in the form of a combined preparation, optionally adapted for a co-treatment with radiotherapy or radio-immunotherapy, to a person in need of such treatment.

The present invention provides also a method for the treatment of diseases involving cell proliferation, migration or apoptosis of myeloma cells, or angiogenesis, which method comprises a simultaneous, separate or sequential co-treatment with an effective amount of an antagonist of at least one receptor selected from TLK1 and TLK2 or with a polymorph, metabolite or pharmaceutically acceptable salt thereof, and with radiotherapy or radio-immunotherapy.

In one embodiment, the tousled-like kinase inhibitor used in the method in accordance with the present invention is an antagonist of at least one kinase tousled-like kinase.

In one embodiment, the tousled-like kinase inhibitor is selected from specific compounds perphenazine, promazine, promazine hydrochloride, thiethylperazine, thiorodazine, trifluoperazine and pharmaceutically acceptable salts, solvates, prodrugs, metabolites, polymorphs, tautomers, racemates, enantiomers, diastereoisomers or derivatives thereof.

The further chemotherapeutic or naturally occurring, semi-synthetic or synthetic therapeutic agent used in the method in accordance with the present invention can be any available chemotherapeutic or naturally occurring, semi-synthetic or synthetic therapeutic agent, and more particularly the chemotherapeutic agents which are commonly used for the treatment of cancer. In one preferred embodiment, amongst the chemotherapeutic or naturally occurring, semi-synthetic or synthetic therapeutic agents, specific compounds are preferred.

In one embodiment, the disease treated in the method in accordance with the present invention is preferably an oncological disease. In a preferred embodiment, the disease is selected from solid tumors, such as urogenital cancers (such as prostate cancer, renal cell cancers, bladder cancers), gynecological cancers (such as ovarian cancers, cervical cancers, endometrial cancers), lung cancer, gastrointestinal cancers (such as colorectal cancers, pancreatic cancer, gastric cancer, oesophageal cancers, hepatocellular cancers, cholangiocellular cancers), head and neck cancer, malignant mesothelioma, breast cancer, malignant melanoma or bone and soft tissue sarcomas, and haematologic neoplasias, such as multiple myeloma, acute myelogenous leukemia, chronic myelogenous leukemia, myelodysplastic syndrome and acute lymphoblastic leukemia. In a preferred embodiment, the disease is hormone sensitive or hormone refractory prostate cancer, ovarian carcinoma, or small cell lung cancer.

In another embodiment, the disease treated in the method in accordance with the present invention is preferably a non-oncological disease selected from diabetic retinopathy, rheumatoid arthritis or psoriasis.

Thus, the beneficial efficacy of the methods in accordance with the invention are mainly based on the additive and synergistic effects of the combined treatment, or to an improved tolerability of the treatment by the patient due, for example, to the administration of lower doses of the therapeutic agents involved.

A further use is that an induction or reinstatement of the sensitivity towards the chemotherapeutic agent is expected in patients treated with the combination of chemotherapeutic agents for which the sensitivity gets lost in the course of the treatment.

According to the present invention, a synergistic combined preparation is meant to comprise an amount of the selected tousled-like kinase inhibitor, or optionally in form of its tautomers, racemates, enantiomers, diastereomers and the mixtures thereof and optionally in form of the pharmacologically acceptable acid addition salts, solvates, hydrates, polymorphs, physiologically functional derivatives or prodrugs thereof, and an amount of the further chemotherapeutic or naturally occurring, semi-synthetic or synthetic therapeutic agent, and/or radiotherapy or radio-immunotherapy, wherein the amount of the individual therapeutic agents alone is insufficient to achieve the therapeutic effect achieved by the administration of the combination of said therapeutic agents, and wherein the combined effects of the amounts of the therapeutic agents is greater than the sum of the therapeutic effects achievable with the amounts of the individual therapeutic agents.

In another embodiment, the present invention also relates to a pharmaceutical combination for the treatment of diseases in which cell proliferation, migration or apoptosis of myeloma cells, or angiogenesis are involved, comprising a selected specific tousled-like kinase inhibitor and a further chemotherapeutic or naturally occurring, semi-synthetic or synthetic therapeutic agent, and/or radiotherapy or radio-immunotherapy, as a combined preparation for simultaneous, separate or sequential use in treatment of said diseases, optionally together with one or more pharmaceutically acceptable diluents and/or carriers.

In another embodiment, the present invention also relates to a pharmaceutical combination preparation kit for the treatment of diseases involving cell proliferation, migration or apoptosis of myeloma cells, or angiogenesis, comprising a therapeutically effective amount of a selected tousled-like kinase inhibitor, or of a polymorph, metabolite or pharmaceutically acceptable salt thereof, and a therapeutically effective amount of a further chemotherapeutic or naturally occurring, semi-synthetic or synthetic therapeutic agent, characterized in that the tousled-like kinase inhibitor is comprised within a first compartment and the further chemotherapeutic or naturally occurring, semi-synthetic or synthetic therapeutic agent is comprised within a second compartment, such that the administration to a patient in need thereof can be simultaneous, separate or sequential, said combination preparation kit being optionally further adapted for a co-treatment with radiotherapy or radio-immunotherapy.

In another embodiment, the present invention thus also provides for the use of a selected tousled-like kinase inhibitor in combination with a further chemotherapeutic or naturally occurring, semi-synthetic or synthetic therapeutic agent, and/or adapted for a co-treatment with radiotherapy or radio-immunotherapy, for the manufacture of a pharmaceutical combination preparation for the treatment of the diseases or indications mentioned hereinbefore.

Within the meaning of the present invention, effective amounts of therapeutic agents and/or of a therapeutic treatment by radiotherapy or radio-immunotherapy means amounts of the agents and/or of the treatment by radiotherapy or radio-immunotherapy which are effective to achieve a therapeutic effect when used in combination.

In another embodiment, the present invention provides for the use of phenothiazine antipsychotics as inhibitors of DSB repair. In another embodiment, the present invention provides for the use of phenothiazine antipsychotics to potentiate tumor cells killing with radiation therapy and/or radiomimetic therapy.

In another embodiment, the present invention provides for the use of pharmacological agents that are able to inhibit DSB repair, and which therefore prevent or treat conditions that are common in mammals, including humans.

The present invention provides for phenothiazines that inhibit TLK sensitized cell killing with RMT. The inhibitors specifically inhibited the TLK mediated phosphorylation of Rad9 (S328) that is DDR responsive, and impaired recovery from the checkpoint. We further established that the inhibitors act specifically on repair pathways controlled by TLK, and result in specific inhibition of NHEJ (non homologous end joining) in several assays. The phenothiazine antipsychotics that inhibit TLK act to sensitize cell killing with RMT (and IR).

Antipsychotic phenothiazines specifically inhibit TLK at nM concentrations in vitro, and at low, well tolerated (μM) concentrations in cultured cell lines. The inhibitors act specifically on the DSB repair pathways controlled by TLKs. Xenograft studies in SCID/bg mice harboring PC 3 human prostate cancer cells or MDA231 Luc human breast cancer cells were undertaken to test the in vivo effects of one selected TLK inhibitor, Thioridazine (THD), with or without doxo, and showed synthetic effects.

The use of adjuvant doxorubicin, at low doses and with low overall toxicity, in combination with TLK inhibitors, improves outcome post-surgery

The present invention also relates to the field of protecting against, or rectifying the effects of DNA damaging agents such as ionizing irradiation.

The present invention is also directed to a method of treating prophylactically or therapeutically body damage resulting from exposure to ionizing radiation and improving patient survival. The method of treatment involves oral administration of a tousled-like kinase activator, alone or in combination with other treatments (for example, other radioprotective agents).

In another embodiment, one or more phenothiazine antipsychotic is administered to a subject that has or will be subjected to DNA damaging agents or irradiation.

In another embodiment, the one or more phenothiazine antipsychotic is selected from the group comprising of chlorpromazine, fluphenazine, metaraminol and prochlorperazine.

Another aspect of the invention is the use of specific activators of TLKs. The activators increase the TLK mediated phosphorylation of Rad9 (S328) and improve checkpoint recovery and DSB repair.

In one embodiment, the method of treatment involves administration of a tousled-like kinase activator composition, alone or in combination with other treatments, both in combination with other radio-protective agents and/or the standard of care.

In another embodiment, the method of treatment provides for a direct administration of tousled-like kinase activators to treat damage to salivary ducts and glands caused by local damaging irradiation.

These and other features are explained more fully in the embodiments illustrated below. It should be understood that in general the features of one embodiment also may be used in combination with features of another embodiment and that the embodiments are not intended to limit the scope of the invention.

### DETAILED DESCRIPTION

It is to be understood that other embodiments may be utilized and changes may be made without departing from the scope of the present invention. Also, it is to be understood that the phraseology and terminology used herein are for the purpose of description and should not be regarded as limiting. The use of “including,” “comprising,” or “having” and variations thereof herein is meant to encompass the items listed thereafter and equivalents thereof as well as additional items.

As used in this specification and the appended claims, the singular forms “a”, “an” and “the” can include plural referents unless the content clearly dictates otherwise. Thus, for example, reference to “a component” can include a combination of two or more components; a reference to “containers” can include individual containers, and the like.

Although many methods and materials similar, modified, or equivalent to those described herein can be used in the practice of the present invention without undue experimentation, the preferred materials and methods are described herein. In describing and claiming the present invention, the following terminology will be used in accordance with the definitions set out below.

As used herein the terms “administer”, “administered”, and “administration” of the various substances denote providing an additional amount of the substance into the animal's bloodstream on the indicated days, whether via daily or other injections on those days or by release on those days from a parenterally administered prolonged release delivery system (e.g., pellet, liquid depot, vaginal suppository or the like), or by continuous dosing (e.g., by an infusion pump) of the substance, delivered parenterally at the beginning of the time period, or, in the case of the continuous dose, throughout the time period. Alternatively it may refer to the delivery of the dosage by periodic (e.g. daily) parenteral injection or implantation or the like.

As used herein, “amelioration” or lessening of the symptoms of a particular symptom, disorder or condition by administration of a particular compound or pharmaceutical composition refers to any decrease of severity, delay in onset, slowing of progression, or shortening of duration, whether permanent or temporary, lasting or transient that is attributed to or associated with administration of the compound or composition.

The term “cancer” includes solid tumors such as breast, ovarian, prostate, lung, kidney, gastric, colon, testicular, head and neck, pancreas, brain, melanoma, and other tumors of tissue organs and cancers of the blood cells, such as lymphomas and leukemias, including acute myelogenous leukemia, chronic lymphocytic leukemia, T cell lymphocytic leukemia, and B cell lymphomas.

A “cellular proliferative disorder” includes those disorders that affect cell proliferation, activation, adhesion, growth, differentiation, or migration processes. As used herein, a “cellular proliferation, activation, adhesion, growth, differentiation, or migration process” is a process by which a cell increases in number, size, activation state, or content, by which a cell develops a specialized set of characteristics which differ from that of other cells, or by which a cell moves closer to or further from a particular location or stimulus. Disorders characterized by aberrantly regulated growth, activation, adhesion, differentiation, or migration cell proliferative disorders” include autoimmune diseases and inflammation for example, an inflammatory or immune system disorder, and/or a cellular proliferative disorder.

The term “DNA-Damaging Treatment” includes treatments that cause DNA damage and have been used extensively include what are commonly known as gamma-rays, X-rays, microwaves, electronic emissions, and/or the directed delivery of radioisotopes to tumor cells. It is most likely that these factors inflict a broad range of damages on DNA and affect DNA replication, gene expression and the assembly and maintenance of chromosomes. Dosage ranges for X-rays range from daily doses of 0.25 to 2.5 Gy for prolonged periods of time (3 to 4 weeks) to single doses of 20 to 60 Gy. Dosage ranges for radioisotopes vary widely, and depend on the half-life of the isotope, the strength and type of radiation emitted, and the uptake by the neoplastic cells. Other agents that damage DNA include compounds also described as “Chemotherapeutic agents”. Agents such as cisplatin, and other DNA alkylating drugs may be used. Agents that damage DNA also include compounds that interfere with DNA replication, mitosis, and chromosomal segregation. Example of these compounds includes etoposide (VP-16), camptothecin and adriamycin, also known as doxorubicin, and the like. Widely used in clinical setting for the treatment of neoplasms these compounds are administered via injection intravenously at doses ranging from 25-75 mg/m2 at 21 day intervals for adriamycin, to 35-50 mg/m2 intravenously or double the intravenous dose orally.

An “effective amount” of a composition disclosed herein is an amount effective to achieve a desired pharmacologic effect or therapeutic improvement without undue adverse side effects. It is understood that “an effective amount” or “a therapeutically effective amount” varies, in some embodiments, from subject to subject, due to variation in metabolism of the compound administered, age, weight, general condition of the subject, the condition being treated, the severity of the condition being treated, and the judgment of the prescribing physician.

The term “flavonol” means a class of flavonoids that have the 3-hydroxyflavone backbone (IUPAC name: 3-hydroxy-2-phenylchromen-4-one). Particular flavanols include 3-hydroxyflavone, azaleatin, fisetin, galangin, gossypetin, kaempferide, kaempferol, isorhamnetin, morin, myricetin, natsudaidain, pachypodol, quercetin, rhamnazin and rhamnetin.

The term “inhibiting” includes preventing, slowing, or reversing the development of a condition, for example, cancer therapy, or advancement of a condition in a patient necessitating treatment.

The term “ionizing radiation”, as used herein, is meant to include, for example, x-rays, gamma rays, cosmic rays, beta particles, alpha particles, high-energy heavier nuclei, high-energy protons, fast electrons, positrons, and solar particles. The exposure to ionizing radiation can be the result of a variety of activities, such as exposures due to high altitude flight, space travel, radiation therapy, accidents, and the like.

The term “pharmaceutically” or “pharmacologically acceptable”, as used herein, refer to molecular entities and compositions that do not produce adverse, allergic, or other untoward reactions when administered to an animal or a human.

The term “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term “pharmaceutically acceptable derivatives” of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs. In addition, a single-isomer formulation of a racemic compound is also a “pharmaceutically acceptable derivative.”

Pharmaceutically acceptable salts include, but are not limited to, amine salts, including but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. The pharmaceutically acceptable salts also include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. The pharmaceutically acceptable salts of the compounds useful in the present invention can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred.

Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl or heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl or heterocyclyl. Pharmaceutically acceptable solvates and hydrates are complexes of a compound with one or more solvent or water molecules, or 1 to about 100, or 1 to about 10, or one to about 2, 3 or 4, solvent or water molecules.

The term “phenothiazine” refers to a heterocyclic structure comprising a central 1,4-thiazine six-membered ring with two additional six-membered aromatic carbon rings symmetrically joined at the 1,3- and 5,6-positions. The term “phenothiazine antipsychotic” refers to a classical antipsychotic that contains a phenothiazine structure. Exemplary phenothiazine antipsychotics include, but are not limited to, acetophenazine, catechin hydrate, ceflazolin sodium salt, chlorpromazine, dienestrol, diethylstilbestrol, equilin, ethoxyquin, fluphenazine, geisemine, luteolin, mesoridazine, mesoridazine, methimazole, methotrimeprazine, perphenazine, phenazopyridine hydrochloride, prochlorperazine, promazine, promazine hydrochloride, promethazine, raloxifene hydrochloride, thiethylperazine, thioridazine, thiorodazine, trifluoperazine and triflupromazine.

The term “phenothiazine derivative” includes, prodrugs (e.g., lipid conjugates, esters), pharmaceutically acceptable salts, solvates, isomers, tautomers, metabolites, analogs, or combinations thereof. In some embodiments, compounds described herein are prepared as prodrugs. A “prodrug” refers to an agent that is converted into the parent drug in vivo. Prodrugs are often useful because, in some situations, they are easier to administer than the parent drug. In some other embodiments, the prodrug is bioavailable whereas the parent is not. In other embodiments, the prodrug also has improved solubility in pharmaceutical compositions over the parent drug. An example, without limitation, of a prodrug would be a compound described herein, which is administered as an ester (the “prodrug”) to facilitate transmittal across a cell membrane where water solubility is detrimental to mobility but which then is metabolically hydrolyzed to the active entity, once inside the cell where water-solubility is beneficial. In other embodiments, the prodrug is a short peptide (e.g., polyaminoacid) bonded to an acid group where the peptide is metabolized to reveal the active moiety. In certain embodiments, the prodrug is a lipid conjugate that aids transport across a biological membrane and is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

To produce a phenothiazine derivative, a pharmaceutically active compound is modified such that the active compound will be regenerated upon in vivo administration. In other embodiments, the derivative is designed to alter the metabolic stability or the transport characteristics of a drug, to mask side effects or toxicity, to improve the flavor of a drug or to alter other characteristics or properties of a drug. In some embodiments phenothiazine derivatives are prodrugs that are designed as reversible drug derivatives, for use as modifiers to enhance drug transport to site-specific tissues. In some embodiments, the design of a prodrug increases the effective water solubility.

In some embodiments, certain sites (e.g. aromatic rings) on the compounds described herein are susceptible to various metabolic reactions, therefore incorporation of appropriate substituents, such as, by way of example only, halogens can reduce, minimize or eliminate this metabolic pathway.

The term “phenothiazine structure” refers to a heterocyclic structure comprising a central 1,4-thiazin six-membered ring with two additional six-membered aromatic carbon rings symmetrically joined at the 1,3- and 5,6-positions. Typically phenothiazine antipsychotics with the phenothiazine structure are substituted at N-10 by a chain having a terminal tertiary amine group 2-3 atoms distant.

A “prodrug” refers to a compound or agent that is converted into the parent drug in vivo. In certain embodiments, a prodrug is enzymatically metabolized by one or more steps or processes to the biologically, pharmaceutically or therapeutically active form of the compound. To produce a prodrug, a pharmaceutically active compound is modified such that the active compound will be regenerated upon in vivo administration. In one embodiment, the prodrug is designed to alter the absorption and/or the transport characteristics of a drug, or to alter other characteristics or properties of a drug. Phenothiazines described herein, in some embodiments, are derivatized into suitable prodrugs.

“Protecting”, as used in the context of ionizing radiation, is meant to refer to any measurable or otherwise observable reduction in one or more of the harmful effects of ionizing radiation. Such reduction in a harmful effect can be ascertained directly, e.g., by monitoring DNA or other cellular changes, or indirectly, by qualitatively or quantitatively evaluating a subject's symptoms resulting from ionizing radiation exposure. As indicated above, the protection need not be and, in many cases, will not be a complete (100%) reduction in the harmful effects of ionizing radiation. For the purposes of illustration, any reduction in any one (or two or three or more) of the harmful effects of ionizing radiation is to be construed as “protecting” the subject from harmful effects of ionizing radiation. Such reduction can be observed in terms of the severity of the harmful effect, the duration of the harmful effect, or both; and, as mentioned above, it can be qualitative or quantitative. Examples of harmful effects of ionizing radiation from which a subject can be protected in accordance with the method of the present invention include: radiation sickness, hair loss (alopecia), weakness, fatigue, nausea, vomiting, diarrhea, skin burns, gastrointestinal tract bleeding, mucous membrane bleeding, gastrointestinal sloughing, oral mucosal sloughing, genetic defects, hematopoietic and/or immunocompetent cell destruction, sterility, bone marrow cancer and other kinds of cancer, premature aging, death, venoocclusive disease of the liver, chronic vascular hyperplasia of cerebral vessels, cataracts, and pneumonites.

Radiation therapy, radio-immunotherapy or pre-targeted radioimmunotherapy are used for the treatment of diseases of oncological nature. “Radiotherapy”, or radiation therapy, means the treatment of cancer and other diseases with ionizing radiation. Ionizing radiation deposits energy that injures or destroys cells in the area being treated (the target tissue) by damaging their genetic material, making it impossible for these cells to continue to grow. Radiotherapy may be used to treat localized solid tumors, such as cancers of the skin, tongue, larynx, brain, breast, lung or uterine cervix. It can also be used to treat leukemia and lymphoma, i.e. cancers of the blood-forming cells and lymphatic system, respectively. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons are machines that produce x-rays of increasingly greater energy. The use of machines to focus radiation (such as x-rays) on a cancer site is called external beam radiotherapy Gamma rays are another form of photons used in radiotherapy Gamma rays are produced spontaneously as certain elements (such as radium, uranium, and cobalt 60) release radiation as they decompose, or decay. Another technique for delivering radiation to cancer cells is to place radioactive implants directly in a tumor or body cavity. This is called internal radiotherapy. Brachytherapy, interstitial irradiation, and intracavitary irradiation are types of internal radiotherapy. In this treatment, the radiation dose is concentrated in a small area, and the patient stays in the hospital for a few days. Internal radiotherapy is frequently used for cancers of the tongue, uterus, and cervix. A further technique is intra-operative irradiation, in which a large dose of external radiation is directed at the tumor and surrounding tissue during surgery. Another approach is particle beam radiation therapy. This type of therapy differs from photon radiotherapy in that it involves the use of fast-moving subatomic particles to treat localized cancers. Some particles (neutrons, pions, and heavy ions) deposit more energy along the path they take through tissue than do x-rays or gamma rays, thus causing more damage to the cells they hit. This type of radiation is often referred to as high linear energy transfer (high LET) radiation. Radio-sensitizers make the tumor cells more likely to be damaged, and radio-protectors protect normal tissues from the effects of radiation. Hyperthermia, the use of heat, may also be used for sensitizing tissue to radiation. Another option involves the use of radio-labeled antibodies to deliver doses of radiation directly to the cancer site (radio-immunotherapy). There are numerous methods available in the art to link a radioisotope to an antibody. For example, for the radio-iodination of the antibody, a method as disclosed in WO 93/05804 may be employed. Another option is to use a linker molecule between the antibody and the radioisotope, e.g. MAG-3 (U.S. Pat. No. 5,082,930, EP 0 247 866), MAG-2 GABA (U.S. Pat. No. 5,681,927, EP 0 284 071), and N2S2 (phenthioate, U.S. Pat. No. 4,897,255, U.S. Pat. No. 5,242,679, EP 0 188 256). A further option is pre-targeted radio-immunotherapy, which may be used to minimize the radiation toxicity by separating the long-circulating antibody and the rapidly cleared radionuclide (Drugs of the future 2003, 28(2), pp. 167-173). Detailed protocols for radiotherapy are readily available to the expert (Cancer Radiotherapy: Methods and Protocols (Methods in Molecular Medicine), Huddart R A Ed., Human Press 2002). The expert knows how to determine an appropriate dosing and application schedule, depending on the nature of the disease and the constitution of the patient. In particular, the expert knows how to assess dose-limiting toxicity (DLT) and how to determine the maximum tolerated dose (MTD) accordingly.

As used herein, the term “subject” is used to mean an animal, preferably a mammal, including a human or non-human. The terms patient and subject may be used interchangeably. The subject is suffering from or at risk of developing cancer or a cell proliferative disorder. Subjects suffering from or at risk of developing cancer or a cell proliferative disorder are identified by methods known in the art.

As used herein, “therapeutically effective amount of a compound” means an amount that is effective to exhibit therapeutic or biological activity at the site(s) of activity in a ruminant, without undue adverse side effects (such as undue toxicity, irritation or allergic response), commensurate with a reasonable benefit/risk ratio when used in the manner of the present invention.

The term “tousled-like kinases” or “TLK”, as used herein, means the nuclear serine/threonine kinases that are involved in the regulation of chromatin assembly. They are rapidly and transiently inhibited by phosphorylation following the generation of DNA double-stranded breaks during S-phase. The TLK1 protein contains a protein kinase ATP-binding motif. TLK1 is expressed in almost all tissues. It is highly expressed is detected in testis. The 787-amino acid protein TLK1 has a 5-domain structure, with N-terminal nuclear localization signals followed by a nucleotide binding motif, and a single catalytic domain near the C terminus. It shares 86% sequence identity with TLK2 overall, and 94% identity in the catalytic region. TLK1 is localized in the nucleus.

### Specific Embodiments

Tumor cell resistance to chemotherapeutic drugs and radiation represents a major problem in clinical oncology. Higher doses of drugs or ionizing radiation may improve the response rate in some malignancies, but these treatment methods also cause increased toxicity in the host. This is particularly true in cases of prostate cancer where the majority of prostate cancer cells are not actively proliferating and are thus resistant to standard cytotoxic therapies. Furthermore, these current methods of cancer therapy, such as radiation therapy and chemotherapy, are either not effective against human prostate cancer or are not specific for prostate carcinoma cells. Accordingly, there is a need in the art for novel methods of treating cancer, in particular, prostate cancer by molecularly targeting drugs that offer the potential of enhancing tumor cell responses to genotoxic treatments while minimizing side effects associated with toxicity.

To that end, the present inventors have discovered a method of treating cancer comprising a combination therapy that utilizes tissue-specific and treatment-specific therapies for cancer. Specifically, the present invention provides a novel method comprising a TLK inhibitor, combined with the administration of potent DNA-damaging agents.

It is another object of the invention to provide a method for treating cancer comprising sensitizing cancer cells to DNA-damaging therapies by administering to a host a therapeutically effective amount of a TLK inhibitor composition and killing the targeted cancer cells by inducing DNA damage and apoptosis.

DNA-damaging agents or factors are defined herein as any chemical compound or treatment method that induces DNA damage when applied to a cell. Such agents and factors include radiation and waves that induce DNA damage, such as irradiation, X-rays, microwaves, electronic emissions, and the like. A variety of chemical compounds, also described as “Chemotherapeutic agents” function to induce DNA damage, all of which are included to be of use in the combined treatment methods disclosed herein. Chemotherapeutic agents contemplated to be of use, include alkylating agents (e.g. cisp-diamine dichloroplatinum (CDDP) or melphalan), agents that interfere with DNA replication, mitosis, and chromosomal segregation (e.g. etoposide (VP-16), camptothecin and adriamycin, also known as doxorubicin), radiomimetic agents (e.g. bleomycin). In certain embodiments, the use of .gamma.-irradiation in combination with a PARP-DBD expression in prostate is particularly preferred as this compound.

As described herein, the diseases which can be treated by the combination in accordance with the present invention are all kind of diseases in which cell proliferation, migration or apoptosis of myeloma cells, or angiogenesis are involved, which can be of oncological nature such as all types of malignant neoplasias or cancers, or of non-oncological nature, such as diabetic retinopathy, rheumatoid arthritis, or psoriasis. Among cancers, selected specific target indications are solid tumors, such as urogenital cancers (such as prostate cancer, renal cell cancers, bladder cancers), gynecological cancers (such as ovarian cancers, cervical cancers, endometrial cancers), lung cancer, gastrointestinal cancers (such as colorectal cancers, pancreatic cancer, gastric cancer, oesophageal cancers, hepatocellular cancers, cholangiocellular cancers), head and neck cancer, malignant mesothelioma, breast cancer, malignant melanoma or bone and soft tissue sarcomas, and haematologic neoplasias, such as multiple myeloma, acute myelogenous leukemia, chronic myelogenous leukemia, myelodysplastic syndrome and acute lymphoblastic leukemia.

The combination treatment in accordance with the present invention is especially efficient for inhibiting tumor growth, survival and metastasis.

Of special interest for the combination treatment is the treatment of hormone sensitive or hormone refractory prostate cancer, ovarian carcinoma, non-small cell lung cancer, small cell lung cancer, or multiple myeloma.

A high percentage of human tumors, including cancer of the prostate (CaP) and breast (BCA), show mutations in DNA repair genes and checkpoint functions that make them overly dependent on alternative pathways for survival. Unfortunately, this can result in carcinomas that are highly resistant to radiation therapy (XRT) or radiomimetic therapy (RMT) from failsafe repair mechanisms also designed to contain excessive genomic instability. Targeting those mechanisms can result in highly specific and effective therapies. The use of inhibitors of TLKs of the provided herein are used to enhance response to radio-chemotherapy will greatly benefit CaP and BCA patients' therapy management.

As already mentioned hereinbefore, the selected tousled-like kinase inhibitors that can be used in the context of the present invention include all substances that inhibit the stimulation or activation of a tousled-like kinase activity.

By inhibition of stimulation or activation of tousled-like kinase is meant any decrease in the activation of the kinase, which need not completely prevent or stop activation of the kinase.

In one embodiment, inhibition of the receptor stimulation or activation, as defined by the present invention, means inhibition resulting from interaction of the antagonist and the receptor or its ligand. By interaction is meant sufficient physical or chemical interaction between the antagonist and the receptor, such that tousled-like kinase activity is inhibited. One of skill in the art would appreciate that examples of such chemical interactions, which include association or bonding, are known in the art and include covalent bonding, ionic bonding, hydrogen bonding, etc., between the antagonist and the receptor or its ligand.

In another embodiment in accordance with the present invention, the selected tousled-like kinase inhibitor binds directly to the receptor. The antagonist can bind externally to the extra-cellular portion of the receptor, which may or may not inhibit binding of the ligand, or internally to the tousled-like kinase domain. Examples of such antagonists include, without limitation, biological molecules, such as antibodies (and functional equivalents thereof) specific for the receptor, and synthetic kinase inhibitors that act directly on the cytoplasmic domain of the receptor.

Additional tousled-like kinase inhibitors can easily be determined using well-known methods. The selected receptor agonists and antagonists to be used in the present invention inhibit the tousled-like kinase activity of the receptor, which generally involves phosphorylation events. Accordingly, phosphorylation assays may for example be useful in determining antagonists useful in the context of the present invention. In addition, methods specific for detection of the receptor expression can be utilized. These include immunohistochemistry for detection of protein expression, fluorescence in situ hybridization for detection of gene amplification, competitive radioligand binding assays, solid matrix blotting techniques, such as Northern and Southern blots, reverse transcriptase polymerase chain reaction and ELISA.

In another embodiment, the one or more tousled-like kinase inhibitor is selected from the group comprising of compounds having a structure:

| Molecule | | CXSMILES |
|---|---|---|
| Name | Structure | (CDD Compatible) |
| LS- 0101778 | 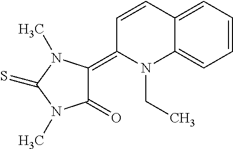 [SMILES: CCN1/C(=C2\C(=O)N(C)C(=S)N2C)C=Cc2ccccc21] | CCN1\C(C═CC2═CC═CC═C12)═C1\N(C)C(═S)N(C)C1═O \|c: 4, 8, t: 6, 10\| |
| LS- 0101786 | 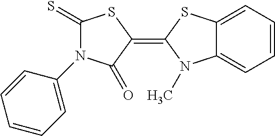 [SMILES: CN1/C(=C2/SC(=S)N(c3ccccc3)C2=O)Sc2ccccc21] | CN1\C(SC2═CC═CC═C12)═C1\SC(═S)N(C1═O)C1═CC═CC═C1 \|c: 6, 22, 24, t: 4, 8, 20\| |
| LS- 0101808 | 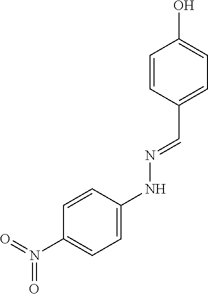 [SMILES: O=[N+]([O-])c1ccc(N/N=C/c2ccc(O)cc2)cc1] | OC1═CC═C(\C═N\NC2═CC═C(C═C2)N(═O)═O)C═C1 \|c: 10, 12, 18, t: 1, 3, 8\| |
| LS- 0101820 | 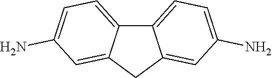 [SMILES: Nc1ccc2c(c1)Cc1cc(N)ccc1-2] | NC1═CC═C2C(CC3═CC(N)═CC═C23)═C1 \|c: 3, 10, 15, t: 1, 7, 12\| |
| LS- 0102608 | 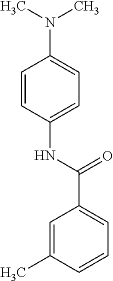 [SMILES: Cc1cccc(C(=O)Nc2ccc(N(C)C)cc2)c1] | CN(C)C1═CC═C(NC(═O)C2═CC═CC(C)═C2)C═C1 \|c: 12, 15, 18, t: 3, 5, 10\| |
| LS- 0102633 | 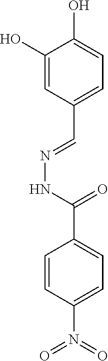 [SMILES: O=C(N/N=C/c1ccc(O)c(O)c1)c1ccc([N+](=O)[O-])cc1] | OC1═CC═C(\C═N\NC(═O)C2═CC═C(C═C2)N(═O)═O)C═C1O \|c: 12, 14, 20, t: 1, 3, 10\| |
| LS- 0102634 | 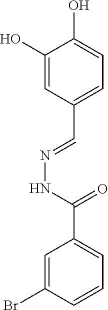 [SMILES: O=C(N/N=C/c1ccc(O)c(O)c1)c1cccc(Br)c1] | OC1═CC═C(\C═N\NC(═O)C2═CC═CC(Br)═C2)C═C1O \|c: 12, 15, 18, t: 1, 3, 10\| |
| LS- 0102802 | 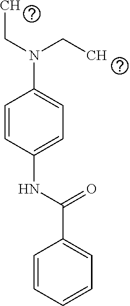 [SMILES: CCN(CC)c1ccc(NC(=O)c2ccccc2)cc1] | CCN(CC)C1═CC═C(NC(═O)C2═CC═CC═C2)C═C1 \|c: 14, 16, 19, t: 5, 7, 12\| |
| LS- 0103445 | 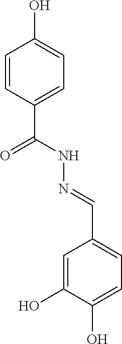 [SMILES: O=C(N/N=C/c1ccc(O)c(O)c1)c1ccc(O)cc1] | OC1═CC═C(C═C1)C(═O)N\N═C\C1═CC═C(O)C(O)═C1 \|c: 3, 5, 19, t: 1, 13, 15\| |
| LS- 0103446 | 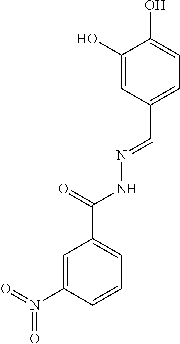 [SMILES: O=C(N/N=C/c1ccc(O)c(O)c1)c1cccc([N+](=O)[O-])c1] | OC1═CC═C(\C═N\NC(═O)C2═CC═CC(═C2)N(═O)═O)C═C1O \|c: 12, 14, 20, t: 1, 3, 10\| |
| LS- 0103511 | 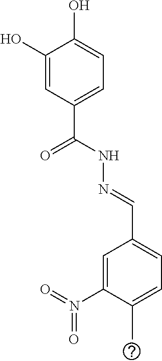 [SMILES: Cc1ccc(/C=N/NC(=O)c2ccc(O)c(O)c2)cc1[N+](=O)[O-]] | OC1═CC═C(C═C1O)C(═O)N\N═C\C1═CC═C(Cl)C(═C1)N(═O)═O \|c: 3, 5, 19, t: 1, 14, 16\| |
| LS- 0103514 | 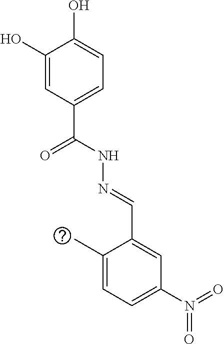 [SMILES: Cc1ccc([N+](=O)[O-])cc1/C=N/NC(=O)c1ccc(O)c(O)c1] | OC1═CC═C(C═C1O)C(═O)N\N═C\C1═CC(═CC═C1Cl)N(═O)═O \|c: 3, 5, 16, 18, t: 1, 14\| |
| LS- 0103572 | 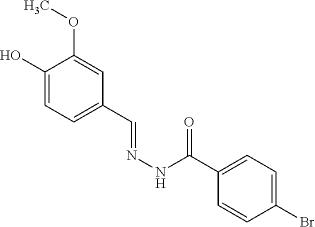 [SMILES: COc1cc(/C=N/NC(=O)c2ccc(Br)cc2)ccc1O] | COC1═CC(\C═N\NC(═O)C2═CC═C(Br)C═C2)═CC═C1O \|c: 15, 17, 19, t: 2, 10, 12\| |
| LS- 0103982 | 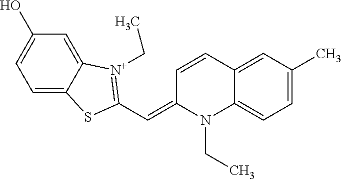 [SMILES: CCN1/C(=C/c2sc3ccc(O)cc3[n+]2CC)C=Cc2cc(C)ccc21] | CCN1\C(═C\C2═[N+](CC)C3═CC(O)═CC═C3S2)C═CC2═CC(C)═CC═C12 \|c: 5, 12, 14, 19, 24, t: 9, 21, 26\| |
| LS- 0104455 | 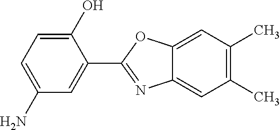 [SMILES: Cc1cc2nc(-c3cc(N)ccc3O)oc2cc1C] | CC1═C(C)C═C2N═C(OC2═C1)C1═CC(N)═CC═C1O \|c: 1, 6, 10, 16, 18, t: 4, 13\| |
| LS- 0104526 | 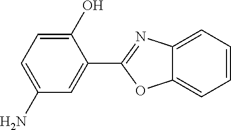 [SMILES: Nc1ccc(O)c(-c2nc3ccccc3o2)c1] | NC1═CC═C(O)C(═C1)C1═NC2═CC═CC═C2O1 \|c: 6, 13, 15, t: 1, 3, 9, 11\| |
| L8- 0104527 | 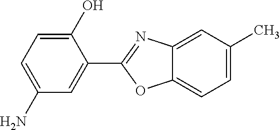 [SMILES: Cc1ccc2oc(-c3cc(N)ccc3O)nc2c1] | CC1═CC═C2OC(═NC2═C1)C1═CC(N)═CC═C1O \|c: 6, 9, 15, 17, t: 1, 3, 12\| |
| LS- 0104848 | 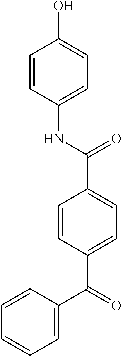 [SMILES: O=C(Nc1ccc(O)cc1)c1ccc(C(=O)c2ccccc2)cc1] | OC1═CC═C(NC(═O)C2═CC═C(C═C2)C(═O)C2═CC═CC═C2)C═C1 \|c: 10, 12, 19, 21, 24, t: 1, 3, 8, 17\| |
| LS- 0104916 | 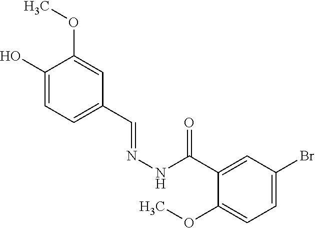 [SMILES: COc1cc(/C=N/NC(=O)c2cc(Br)ccc2OC)ccc1O] | COC1═CC(\C═N\NC(═O)C2═CC(Br)═CC═C2OC)═CC═C1O \|c: 13, 15, 19, 21, t: 2, 10\| |
| LS- 0104926 | 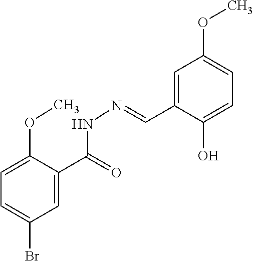 [SMILES: COc1ccc(O)c(/C=N/NC(=O)c2cc(Br)ccc2OC)c1] | COC1═CC═C(O)C(\C═N\NC(═O)C2═CC(Br)═CC═C2OC)═C1 \|c: 16, 18, 22, t: 2, 4, 13\| |
| LS- 0104948 | 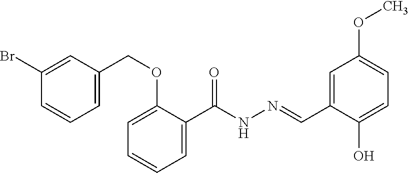 [SMILES: COc1ccc(O)c(/C=N/NC(=O)c2ccccc2OCc2cccc(Br)c2)c1] | COC1═CC═C(O)C(\C═N\NC(═O)C2═CC═CC═C2OCC2═CC═CC(Br)═C2)═C1 \|c: 15, 17, 24, 27, 29, t: 2, 4, 13, 22\| |
| LS- 0105185 | 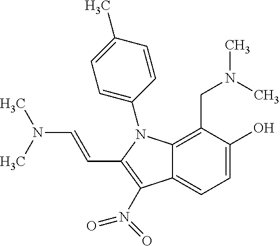 [SMILES: Cc1ccc(-n2c(/C=C/N(C)C)c([N+](=O)[O-])c3ccc(O)c(CN(C)C)c32)cc1] | CN(C)CC1═C2N(C(\C═C\N(C)C)═C(C2═CC═C1O)N(═O)═O)C1═CC═C(C)C═C1 \|c: 4, 12, 15, 17, 29, t: 24, 26\| |
| LS- 0106470 | 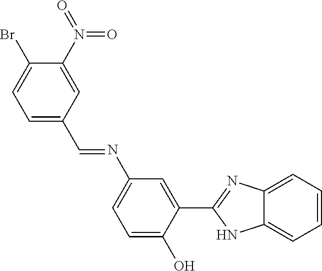 [SMILES: O=[N+]([O-])c1cc(/C=N/c2ccc(O)c(-c3nc4ccccc4[nH]3)c2)ccc1Br] | OC1═CC═C(C═C1C1═NC2═CC═CC═C2N1)\N═C\C1═CC═C(Br)C(═C1)N(═O)═O \|c: 3, 5, 12,14, 26, t: 1, 8, 10, 21, 23\| |
| LS- 0106620 | 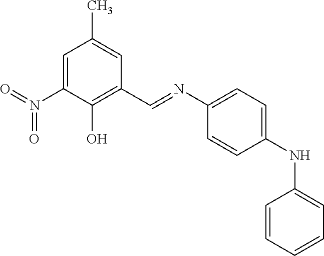 [SMILES: Cc1cc(/C=N/c2ccc(Nc3ccccc3)cc2)c(O)c([N+](=O)[O-])c1] | CC1═CC(\C═N\C2═CC═C(NC3═CC═CC═C3)C═C2)═C(O)C(═C1)N(═O)═O \|c: 13, 15, 18, 23, t: 1, 6, 8, 11, 20\| |
| LS- 0106669 | 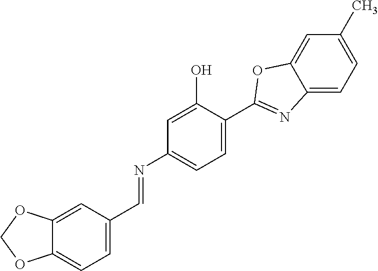 [SMILES: Cc1ccc2nc(-c3ccc(/N=C/c4ccc5c(c4)OCO5)cc3O)oc2c1] | CC1═CC═C2N═C(OC2═C1)C1═CC═C(C═C1O)\N═C\C1═CC═C2OCOC2═C1 \|c: 5, 9, 14, 16 , 30, t: 1, 3, 12, 22, 24\| |
| LS- 0106684 | 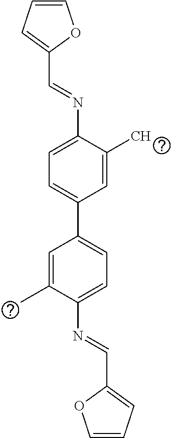 | CC1═CC(═CC═C1\N═C\C1═CC═CO1)C1═CC═C(\N═C\C2═CC═CO2)C(C)═C1 \|c: 3, 5, 12, 24, 29, t: 1, 10, 16, 18, 22\| |
| LS- 0106685 | 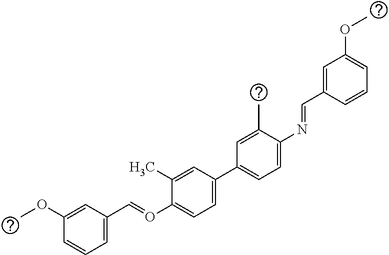 | COC1═CC(\C═N\C2═CC═C(C═C2C)C2═CC═C(\N═C\C3═CC(OC)═CC═C3)C(C)═C2)═CC═C1 \|c: 9, 11, 25, 27, 31, 33, 35, t: 2, 7, 15, 17, 21\| |
| LS- 0106745 | 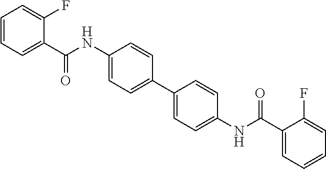 [SMILES: O=C(Nc1ccc(-c2ccc(NC(=O)c3ccccc3F)cc2)cc1)c1ccccc1F] | FC1═CC═CC═C1C(═O)NC1═CC═C(C═C1)C1═CC═C(NC(═O)C2═CC═CC═C2F)C═C1 \|c: 3, 5, 13, 15, 27, 29, 33, t: 1, 11, 18, 20, 25\| |
| LS- 0107100 | 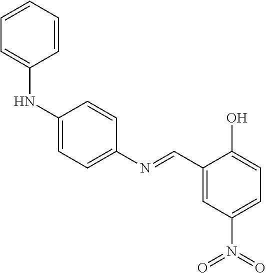 [SMILES: O=[N+]([O-])c1ccc(O)c(/C=N/c2ccc(Nc3ccccc3)cc2)c1] | OC1═CC═C(C═C1\C═N\C1═CC═C(NC2═CC═CC═C2)C═C1)N(═O)═O \|c: 3, 5, 17, 19, 22, t: 1, 10, 12, 15\| |
| LS- 0107128 | 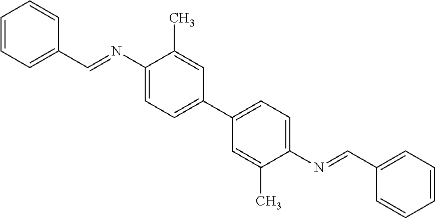 [SMILES: Cc1cc(-c2ccc(/N=C/c3ccccc3)c(C)c2)ccc1/N=C/c1ccccc1] | CC1═CC(═CC═C1\N═C\C1═CC═CC═C1)C1═CC═C(\N═C\C2═CC═CC═C2)C(C)═C1 \|c: 3, 5, 12, 14, 25, 27, 31, t: 1, 10, 17, 19, 23\| |
| LS- 0107148 | 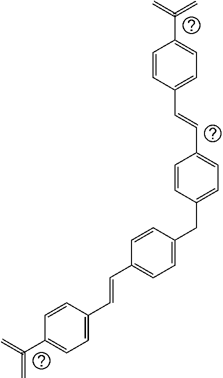 | O═N(═O)C1═CC═C(\C═N\C2═CC═C(SC3═CC═C(C═C3)\N═C\C3═CC═C(C═C3)N(═O)═O)C═C2)C═C1 \|c: 16, 18, 25, 27, 33, 36, t: 3, 5, 9, 11, 14, 23\| |
| LS- 0107187 | 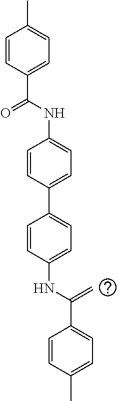 [SMILES: C=C(Nc1ccc(-c2ccc(NC(=O)c3ccc(C)cc3)cc2)cc1)c1ccc(C)cc1] | IC1═CC═C(C═C1)C(═O)NC1═CC═C(C═C1)C1═CC═C(NC(═O)C2═CC═C(I)C═C2)C═C1 \|c: 3, 5, 13, 15, 30, 33, t: 1, 11, 18, 20, 25, 27\| |
| LS- 0108072 | 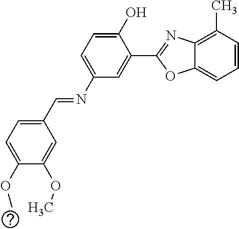 [SMILES: COc1ccc(/C=N/c2ccc(O)c(-c3nc4c(C)cccc4o3)c2)cc1OC] | COC1═CC═C(\C═N\C2═CC═C(O)C(═C2)C2═NC3═C(C)C═CC═C3O2)C═C1OC \|c: 13, 18, 21, 23, 28, t: 2, 4, 8, 10, 16\| |
| LS- 0108075 | 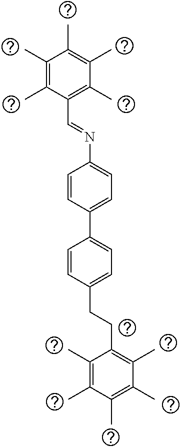 | CC1═C(Cl)C(C)═C(\C═N\C2═CC═C(C═C2)C2═CC═C(C═C2)\N═C\C2═C(C)C(Cl)═C(C)C(Br)═C2O)C(O)═C1Br \|c: 1, 11, 13, 18, 20, 25, 33, 38, t: 5, 9, 16, 29\| |
| LS- 0108128 | 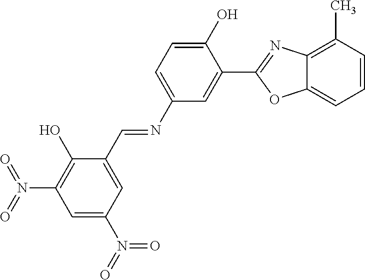 [SMILES: Cc1cccc2oc(-c3cc(/N=C/c4cc([N+](=O)[O-])cc([N+](=O)[O-])c4O)ccc3O)nc12] | CC1═C2N═C(OC2═CC═C1)C1═CC(═CC═C1O)\N═C\C1═CC(═CC(═C1O)N(═O)═O)N(═O)═O \|c: 1, 3, 7, 9, 14, 16, 24, 26, t: 12, 22\| |
| LS- 0108136 | 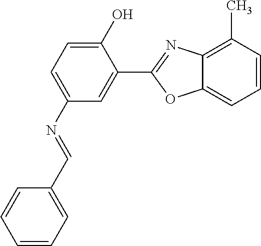 [SMILES: Cc1cccc2oc(-c3cc(/N=C/c4ccccc4)ccc3O)nc12] | CC1═C2N═C(OC2═CC═C1)C1═CC(═CC═C1O)\N═C\C1═CC═CC═C1 \|c: 1, 3, 7, 9, 14, 16, 24, 26, t: 12, 22\| |
| LS- 0108251 | 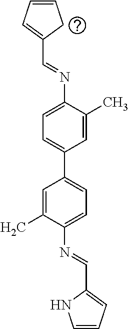 | CC1═CC(═CC═C1\N═C\C1═CC═CN1)C1═CC═C(\N═C\C2═CC═CN2)C(C)═C1 \|c: 3, 5, 12, 24, 29, t: 1, 10, 16, 18, 22\| |
| LS- 0108267 | 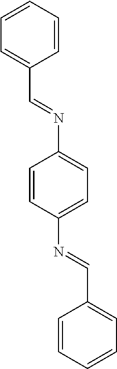 [SMILES: C(=N\c1ccc(/N=C/c2ccccc2)cc1)/c1ccccc1] | C(═N/C1═CC═C(C═C1)\N═C\C1═CC═CC═C1)\C1═CC═CC═C1 \|c: 4, 6, 13, 15, 20, 22, t: 2, 11, 18\| |
| LS- 0108365 | 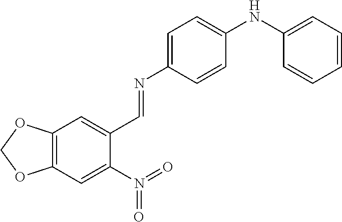 [SMILES: O=[N+]([O-])c1cc2c(cc1/C=N/c1ccc(Nc3ccccc3)cc1)OCO2] | O═N(═O)C1═CC2═C(OCO2)C═C1\C═N\C1═CC═C(NC2═CC═CC═C2)C═C1 \|c: 11, 23, 25, 28, t: 3, 5, 16, 18, 21\| |
| LS- 0108377 | 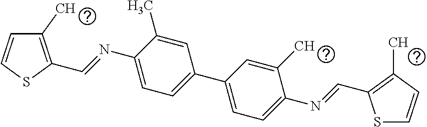 [SMILES: Cc1cc(-c2ccc(/N=C/c3sccc3C)c(C)c2)ccc1/N=C/c1sccc1C] | CC1═C(SC═C1)\C═N\C1═CC═C(C═C1C)C1═CC═C(\N═C\C2═C(C)C═CS2)C(C)═C1 \|c:4, 11, 13, 23, 26, 31, t: 1, 9, 17, 19\| |
| LS- 0108506 | 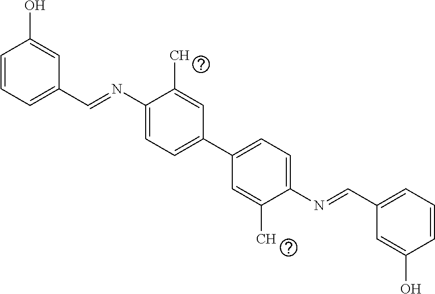 [SMILES: Cc1cc(-c2ccc(/N=C/c3cccc(O)c3)c(C)c2)ccc1/N=C/c1cccc(O)c1] | CC1═CC(═CC═C1\N═C\C1═CC═CC(O)═C1)C1═CC═C(\N═C\C2═CC═CC(O)═C2)C(C)═C1 \|c: 3, 5, 12, 15, 26, 29, 33, t: 1, 10, 18, 20, 24\| |
| LS- 0108531 | 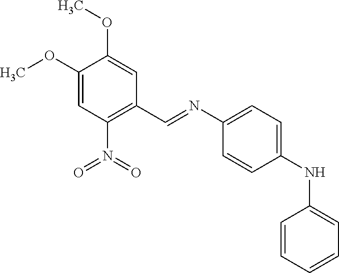 [SMILES: COc1cc(/C=N/c2ccc(Nc3ccccc3)cc2)c([N+](=O)[O-])cc1OC] | COC1═CC(\C═N\C2═CC═C(NC3═CC═CC═C3)C═C2)═C(C═C1OC)N(═O)═O \|c: 14, 16, 19, 21, 23, t: 2, 7, 9, 12\| |
| LS- 0108748 | 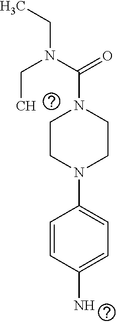 [SMILES: CCN(CC)C(=O)N1CCN(c2ccc(N)cc2)CC1] | CCN(CC)C(═O)N1CCN(CC1)C1═CC═C(N)C═C1 \|c: 19, t: 14, 16\| |
| LS- 0108847 | 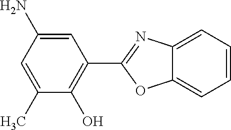 [SMILES: Cc1cc(N)cc(-c2nc3ccccc3o2)c1O] | CC1═CC(N)═CC(C2═NC3═CC═CC═C3O2)═C1O \|c: 4, 11, 13, 17, t: 1, 7, 9\| |
| LS- 0200297 | 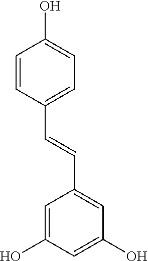 [SMILES: Oc1ccc(/C=C/c2cc(O)cc(O)c2)cc1] | OC1═CC═(\C═C\C2═CC(O)═CC(O)═C2)C═C1 \|c: 10, 13, 16, t: 1, 3, 7\| |
| LS- 0400765 | 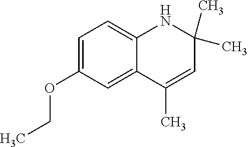 [SMILES: CCOc1ccc2c(c1)C(C)=CC(C)(C)N2] | CCOC1═CC═C2NC(C)(C)C═C(C)C2═C1 \|c: 15, t: 3, 5, 11\| |
| LS- 0700007 | 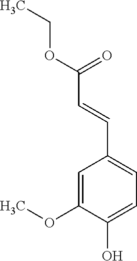 [SMILES: CCOC(=O)/C=C/c1ccc(O)c(OC)c1] | CCOC(═O)\C═C\C1═CC═C(O)C(OC)═C1 \|c: 14, t: 7, 9\| |
| LS- 0700026 | 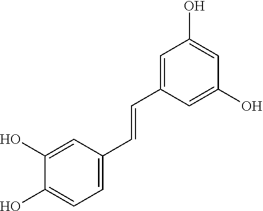 [SMILES: Oc1cc(O)cc(/C=C/c2ccc(O)c(O)c2)c1] | OC1═CC(\C═C\C2═CC═C(O)C(O)═C2)═CC(O)═C1 \|c: 12, 14, 17, t: 1, 6, 8\| |
| LS- 0700040 | 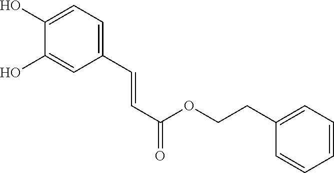 [SMILES: O=C(/C=C/c1ccc(O)c(O)c1)OCCc1ccccc1] | OC1═CC═C(\C═C\C(═O)OCCC2═CC═CC═C2)C═C1O \|c: 14, 16, 19, t: 1, 3, 12\| |
| LS- 0700060 | 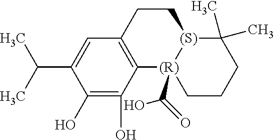 | CC(C)C1═CC2═C(C(O)═C1O)[C@]1(CCCC(C)(C)[C@@H]1CC2)C(O)═O \|c: 5, 8, t: 3\| |
| LS- 0700071 | 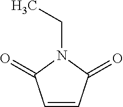 | CCN1C(═O)C═CC1═O \|c: 5\| |
| LS- 0700072 | 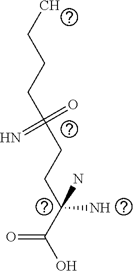 | [H][C@](N)(CCS(═N)(═O)CCCC)C(O)═O |
| LS- 0700084 | 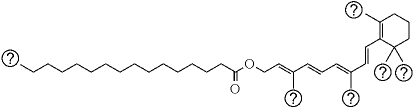 [SMILES: CCCCCCCCCCCCCCCC(=O)OC/C=C(C)/C=C/C=C(C)/C=C/C1=C(C)CCCC1(C)C] | CCCCCCCCCCCCCCCC(═O)OC\C═C(/C)\C═C\C═C(/C)\C═C\C1═C(C)CCCC1(C)C \|c: 29\| |
| LS- 0103440 | 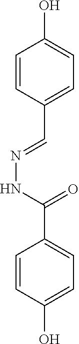 [SMILES: O=C(N/N=C/c1ccc(O)cc1)c1ccc(O)cc1] | Oc1ccc(\C═N\NC(═O)c2ccc(O)cc2)cc1 |
| LS- 0103445 | 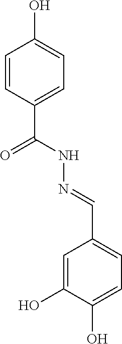 [SMILES: O=C(N/N=C/c1ccc(O)c(O)c1)c1ccc(O)cc1] | Oc1ccc(cc1)C(═O)N\N═C\c1ccc(O)c(O)c1 |
| LS- 0103446 | 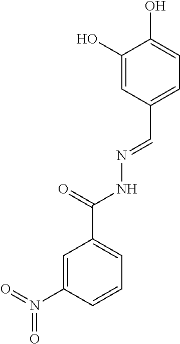 [SMILES: O=C(N/N=C/c1ccc(O)c(O)c1)c1cccc([N+](=O)[O-])c1] | Oc1ccc(\C═N\NC(═O)c2cccc(c2)N(═O)═O)cc1O |
| LS- 0103509 | 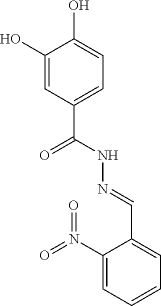 [SMILES: O=C(N/N=C/c1ccccc1[N+](=O)[O-])c1ccc(O)c(O)c1] | Oc1ccc(cc1O)C(═O)N\N═C\c1ccccc1N(═O)═O |
| LS- 0103511 | 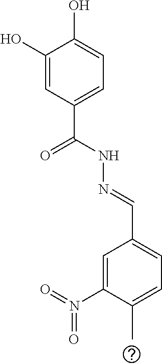 [SMILES: Cc1ccc(/C=N/NC(=O)c2ccc(O)c(O)c2)cc1[N+](=O)[O-]] | Oc1ccc(cc1O)C(═O)N\N═C\c1ccc(Cl)c(c1)N(═O)═O |
| LS- 0103512 | 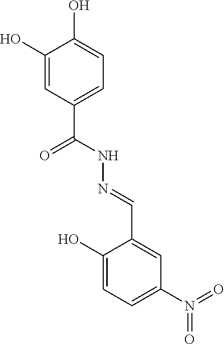 [SMILES: O=C(N/N=C/c1cc([N+](=O)[O-])ccc1O)c1ccc(O)c(O)c1] | Oc1ccc(cc1O)C(═O)N\N═C\c1cc(ccc1O)N(═O)═O |
| LS- 0103514 | 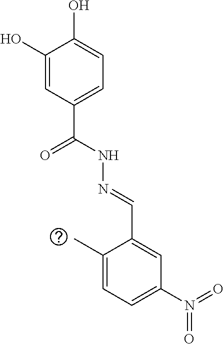 [SMILES: Cc1ccc([N+](=O)[O-])cc1/C=N/NC(=O)c1ccc(O)c(O)c1] | Oc1ccc(cc1O)C(═O)N\N═C\c1cc(ccc1Cl)N(═O)═O |
| LS- 0103572 | 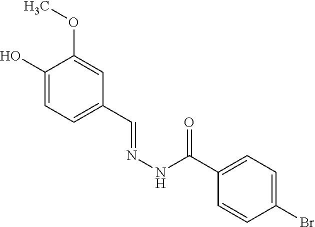 [SMILES: COc1cc(/C=N/NC(=O)c2ccc(Br)cc2)ccc1O] | COc1cc(\C═N\NC(═O)c2ccc(Br)cc2)ccc1O |

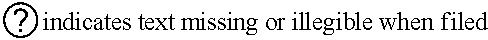

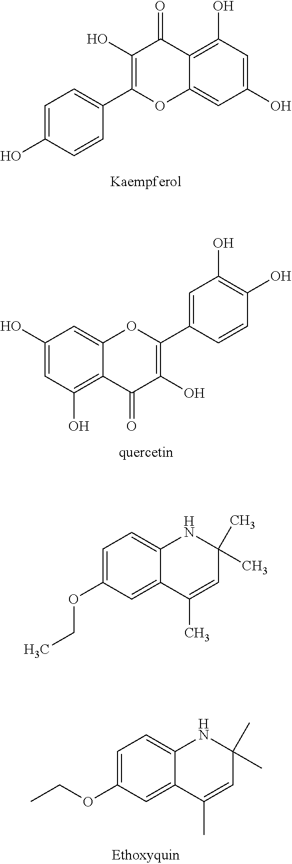

### Use of TLK Activators

In another embodiment of the present invention, compounds described herein are used to activate or stimulate activity in tousled-like kinase.

The present invention also provides for a method of reducing or eliminating the effects of ionizing radiation on normal cells in a subject who has incurred or is at risk for incurring exposure to ionizing radiation, comprising administering to the subject an effective amount of at least one radioprotective tousled-like kinase activating compound to the subject prior to or after exposure to ionizing radiation.

Increased tousled-like kinase stimulation or activation can result from higher levels of ligand, receptor gene amplification, increased transcription of the receptor or mutations that cause unregulated receptor signaling. Amplification of the gene encoding the receptor results in an increased number of ligands binding to the receptor, which can further stimulate cell proliferation. The tousled-like kinase receptor may also be over-expressed through gene amplification or increases in transcription, mRNA translation, or stability of the protein.

The present invention is directed to a method of treating prophylactically or therapeutically body damage resulting from exposure to ionizing radiation and improving patient survival. The method of treatment involves oral administration of a tousled-like kinase activator, alone or in combination with other treatments (for example, other radioprotective agents).

In another embodiment, one or more tousled-like kinase activator is a phenothiazine antipsychotic administered to a subject that has or will be subjected to DNA damaging agents or irradiation.

In another embodiment, the one or more phenothiazine antipsychotic is selected from the group comprising of chlorpromazine, fluphenazine, metaraminoland prochlorperazine.

In another embodiment, the one or more tousled-like kinase activator is selected from the group comprising of compounds having a structure:

| Molecule | | CXSMILES |
|---|---|---|
| Name | Structure | (CDD Compatible) |
| LS-0103552 | 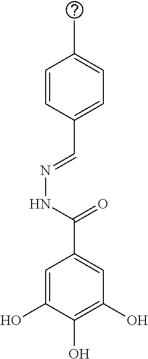 [SMILES: Cc1ccc(/C=N/NC(=O)c2cc(O)c(O)c(O)c2)cc1] | Oc1cccc(\C═N\NC(═O)c2cc(O)c(O)c(O)c2)cc1 |
| LS-0103553 | 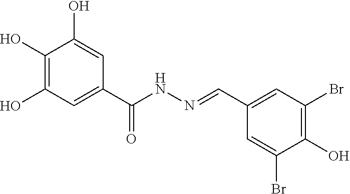 [SMILES: O=C(N/N=C/c1cc(Br)c(O)c(Br)c1)c1cc(O)c(O)c(O)c1] | Oc1cc(cc(O)c1O)C(═O)N\N═C\c1cc(Br)c(O)c(Br)c1 |
| LS-0103554 | 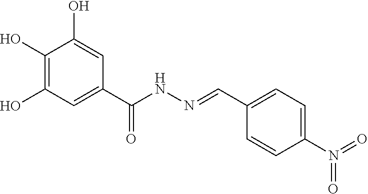 [SMILES: O=C(N/N=C/c1ccc([N+](=O)[O-])cc1)c1cc(O)c(O)c(O)c1] | Oc1cc(cc(O)c1O)C(═O)N\N═C\c1ccc(cc1)N(═O)═O |
| LS-0103555 | 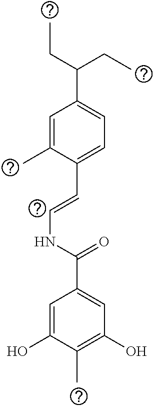 [SMILES: CCC(CC)c1ccc(/C=C/NC(=O)c2cc(O)c(C)c(O)c2)c(C)c1] | CCN(CC)c1ccc(\C═N\NC(═O)c2cc(O)c(O)c(O)c2)c(O)c1 |

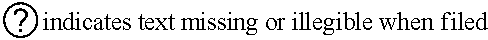

In another embodiment, one or more tousled-like kinase activator is a phenol. In another embodiment, one or more tousled-like kinase activator is a polyphenol. In another embodiment, one or more tousled-like kinase activator is a polyphenol selected from the group consisting of carnosic acid, quercetin, resveratrol, gallic acid, chicoric acid, gingerol and curcumin.

In another embodiment, one or more tousled-like kinase activator is a polyphenol compound selected from the group consisting of (+)-catechin, (+)-gallocatechin, (−)-gallocatechin gallate, (−)-epicatechin, (−)-epicatechin gallate, (−)-epigallocatechin, (−)-catechin gallate, (−)-epigallocatechin gallate, gallic acid, free theaflavin, theaflavin monogallate A, theaflavin monogallate B, and theaflavin digallate.

In another embodiment, one or more tousled-like kinase activator is a polyphenol compound selected from the group consisting of gallic acid, methyl gallate, ethyl gallate, propyl gallate, octyl gallate, p-coumaric acid, caffeic acid, ferulic acid, salicylic acid, sinaptic acid, chlorogenic acid, curcumins, and analogues thereof in which the carboxylic acid group is esterified with a C1-C10 alcohol, and mixtures thereof.

In another embodiment, one or more tousled-like kinase activator is a polyphenol compound selected from the group consisting of phosphorylated forms of catechol, DL-3,4-dihydroxyphenylalanine, DL-DOPA, catecholamines, 3-hydroxytyramine, dopamine, phloroglucinol, phenolic acids, caffeic acid, dihydrocaffeic acid, ferulic acid, protocatechuic acid, chlorogenic acid, isochlorogenic acid, gentisic acid, homogentisic acid, gallic acid, hexahydroxydiphenic acid, ellagic acid, rosmarinic acid, lithospermic acid, curcumin; polyhydroxylated coumarins, polyhydroxylated lignans, neolignans; silymarin, apigenol, luteolol, quercetin, quercetagin, quercetagetin, chrysin, myricetin, rhamnetin, genistein, morin, gossypetin, kaempferol, rutin, naringin, narigenin, hesperitin, hesperidin, diosmin, diosmoside, amentoflavone, fisetin, vitexin, isoliquirtigenin, hesperidin methylchalcone, taxifoliol, silybin, silychristin, silydianin, catechin, epicatechin, gallocatechin, catechin gallate, gallocatechin gallate, epicatechin gallate, epigallocatechin gallate, epigallocatechin, glucogallin; proanthocyanidin, propyl gallate, isoamyloctyl gallate, dodecyl gallate, penta-O-galloyl glucose, tannic acid, gallotannin, ellagitannin, shikimic acid, salicylic acid, resveratrol (3,4′,5′-trihydroxystilbene); plant extracts containing one or more of these compounds; and mixtures thereof.

Treatment with TLK activators can produce beneficial effects for normal tissues and organs exposed to the same genotoxic regimens: XRT, radiomimetic chemotherapy, or even daily skin exposure to UV damage. Both gene therapy approaches aimed at sparing salivary glands from the damaging effects of XRT to treat head and neck cancer, as well as direct TAT-TLK1 protein delivery to salivary glands have been may be used. Additional modes of delivery of these proteins, such as a topical skin delivery in a liposomal complex (of either the protein itself or via viral or plasmid gene delivery vehicle) are options.

In accordance with the present invention, a tousled-like kinase activator provided in any of the above-described pharmaceutical carriers administered to a subject suspected of or having been exposed to irradiation or administered to a subject prior to exposure to irradiation or in anticipation of exposure. One of skill in the art can determine the therapeutically effective amount of phenothiazine antipsychotic to be administered to a subject based upon several considerations, such as absorption, metabolism, method of delivery, age, weight, severity of ionizing damage and response to the therapy. Oral administration of the tousled-like kinase activator includes oral, buccal, enteral or intragastric administration. It is also envisioned that the composition may be used as a food additive. Topical administration of the tousled-like kinase activator includes topical, dermal, epidermal, or subcutaneous administration.

Treatment regimens may vary as well, and often depend on the type of ionizing damage or exposure, location of damage or exposure, disease progression that resulted from damage or exposure, and health and age of the patient. Obviously, certain types of conditions will require more aggressive treatment, while at the same time, certain patients cannot tolerate more taxing protocols. The clinician will be best suited to make such decisions based on the known efficacy and toxicity (if any) of the therapeutic formulations.

Subjects may be exposed to ionizing radiation when undergoing therapeutic irradiation for the treatment of proliferative disorders. Such disorders included cancerous and non-cancer proliferative disorders. For example, the present compounds are believed effective in protecting normal cells during therapeutic irradiation of a broad range of tumor types, including but not limited to the following: breast, prostate, ovarian, lung, colorectal, brain (i.e., glioma) and renal. The compounds are also effective against leukemic cells.

The compounds are also believed useful in protecting normal cells during therapeutic irradiation of abnormal tissues in non-cancer proliferative disorders, including but not limited to the following: hemangiomatosis in new born, secondary progressive multiple sclerosis, chronic progressive myelodegenerative disease, neurofibromatosis, ganglioneuromatosis, keloid formation, Paget's Disease of the bone, fibrocystic disease of the breast, Peronies and Duputren's fibrosis, restenosis and cirrhosis.

According to the invention, therapeutic ionizing radiation may be administered to a subject on any schedule and in any dose consistent with the prescribed course of treatment, as long as the tousled-like kinase activator radioprotectant compound is administered prior to the radiation. The course of treatment differs from subject to subject, and those of ordinary skill in the art can readily determine the appropriate dose and schedule of therapeutic radiation in a given clinical situation.

The tousled-like kinase activator should be administered far enough in advance of the therapeutic radiation such that the compound is able to reach the normal cells of the subject in sufficient concentration to exert a radioprotective effect on the normal cells. The tousled-like kinase activator may be administered as much as about 24 hours, preferably no more than about 18 hours, prior to administration of the radiation. In one embodiment, the tousled-like kinase activator is administered at least about 6-12 hours before administration of the therapeutic radiation. Most preferably, the tousled-like kinase activator is administered once at about 18 hours and again at about 6 hours before the radiation exposure. One or more tousled-like kinase activators may be administered simultaneously, or different tousled-like kinase activators may be administered at different times during the treatment.

Where the therapeutic radiation is administered in serial fashion, it is preferable to intercalate administration of one or more tousled-like kinase activators within the schedule of radiation treatments. As above, different tousled-like kinase activators may be administered either simultaneously or at different times during the treatment. Preferably, an about 24 hour period separates administration of tousled-like kinase activator and the therapeutic radiation. More preferably, the administration of tousled-like kinase activator and the therapeutic radiation is separated by about 6 to 18 hours. This strategy will yield significant reduction in radiation-induced side effects without affecting the anticancer activity of the therapeutic radiation.

The tousled-like kinase activator can be administered orally as a solution in a suitable buffer, or as a solid oral dosage in the form of a capsule, tablet or similar suitable format, or as a topical formulation. The amount of phenothiazine antipsychotic that is administered is from 0.01 to 200.0 g/kg, preferably from 0.01 to 100.0 g/kg, as a single or a divided dose. The treatment is envisaged to continue until the damage has been normalized, preferably for 30 days of continuous treatment. The effect of treatment can be monitored by determining peripheral blood cell composition, in particular the content of white blood cells in circulation, and more generally by the overall physical status of the subjects.

The treatments may include various “unit doses.” Unit dose is defined as containing a predetermined quantity of the therapeutic composition (tousled-like kinase activator) calculated to produce the desired responses in association with its administration, i.e., the appropriate route and treatment regimen. The quantity to be administered, and the particular route and formulation, are within the skill of those in the clinical arts. Also of import is the subject to be treated, in particular, the state of the subject and the protection desired. A unit dose administered i.v. or s.c. need not be administered as a single injection but may comprise continuous infusion over a set period of time.

In specific embodiments, tousled-like kinase activator is given in a single dose or multiple doses. The single dose may be administered daily, or multiple times a day, or multiple times a week. In a further embodiment, the tousled-like kinase activator is given in a series of doses. The series of doses may be administered daily, or multiple times a day, weekly, or multiple times a week.

In one embodiment of the present invention, tousled-like kinase activator is administered in an effective amount to prevent, reduce, decrease, or inhibit the damage caused by irradiation of the body by damaging ionizing radiation and improve patient survival. The amount of phenothiazine antipsychotic that is administered is from 0.01 to 20 g/kg, preferably from 0.01 to 5 g/kg, as a single or a divided dose. The treatment is envisaged to continue until the damage has been normalized, preferably for 30 days of continuous treatment.

The improvement is any observable or measurable change for the better. The composition and the method of treatment of this invention may decrease the mortality of subjects exposed to damaging irradiation. In other aspect, the composition of this invention is administered in an effective amount to decrease, reduce, inhibit, prevent or eliminate damage to, and the loss of function of the cells of the immune system, and the loss of function of the primary physical means of body defense, for example the GI epithelial barrier and salivary glands and ducts. Repeated administration of tousled-like kinase activator can result in the attenuation of the consequences of absorption by the body of a damaging dose of radiation.

Damage to the immune and hematopoietic system following an absorbed dose of damaging irradiation makes subjects susceptible to opportunistic infections and disease. Total leukocyte count is traditionally as an indicator of immune system damage. While all PBMCs (Peripheral Blood Mononuclear Cells) decline in absolute numbers after radiation exposure, some change faster than others, leading to alterations in the proportions of various blood cell populations relative to their original proportions. Thus, it is envisioned that the tousled-like kinase activator of the present invention can be used with compositions that enhance or increase the PBMCs or reduce the attenuation of PBMCs. More specifically it is known that the damage results in changes in the relative composition of immune cells in circulation such as an increase in CD4+ T lymphocytes, decrease in B lymphocytes and a dramatic increase in natural killer cells. Such changes result in immune dysregulation and depressed immune responsiveness to antigenic challenge. Thus, additional immune enhancer agents can correct or positively alter the immune dysregulation that occurs in response to irradiation damage. In further embodiments, cytokines, for example, interleukin-18 or granulocyte/macrophage colony-stimulating factor, can be used to stimulate the production or activity of immune cells.

In order to increase the effectiveness of the tousled-like kinase activator of the present invention, it may be desirable to combine the composition of the present invention with other agents effective in providing protection or treating ionizing radiation. These other radioprotective compositions would be provided in a combined amount effective to promote therapeutic benefit. This process may involve administering the tousled-like kinase activator of the present invention and the agent(s) or multiple factor(s) at the same time. This may be achieved by administering a single composition or pharmacological formulation that includes both agents, or by administering two distinct compositions or formulations, at the same time, or at times close enough so as to result in an overlap of this effect, wherein one composition includes the tousled-like kinase activator and the other includes the second agent(s).

Alternatively, the tousled-like kinase activator of the present invention may precede or follow the other radioprotective agent and/or treatment by intervals ranging from minutes to weeks. In embodiments where the radioprotective agent and tousled-like kinase activator are administered or applied separately, one would generally ensure that a significant period of time did not expire between the time of each delivery, such that the agent and tousled-like kinase activator would still be able to exert an advantageously combined effect. In such instances, it is contemplated that one may contact the area and/or administer to the subject to be treated both modalities within about 1-14 days of each other and, more preferably, within about 12-24 hours of each other. In some situations, it may be desirable to extend the time period for treatment significantly, however, where several days (2, 3, 4, 5, 6 or 7) to several weeks (2, 3, 4, 5, 6, 7 or 8) lapse between the respective administrations.

In specific embodiment, treatment with phenothiazine antipsychotic can be combined with other treatments aiming to lessen the effects of damaging radiation, for example with granulocyte-stimulating factor (G-CSF) (Filgrastim/(Neupogen)) or with Amifostine, or with other agents intended to treat the consequences of radiation damage.

Examples of thiols that can be used as radioprotective agents include, but are not limited to cysteine, cysteamine, cystamine, AET and 2-mercaptoethylguanidine (MEG). The sulfhydrylamines are also potent agents which reduce temperatures and physiological pH. The dose reduction factor (DRF) of various compounds ranges from 1.4 to 2.0. This class of compounds is characterized by the sulfhydryl compounds (SH) and amine (NH2) separated by 2 carbon atoms. Other —SH radicals that can be used as radioprotective agents include, but are not limited to thiourea, thiouracil, dithiocarbamate, dithioxamides, thiazolines, sulfoxides and sulfones.

Pharmacological agents that can be used as radioprotective agents can include anesthetic drugs and alchohol, analgesics (e.g., morphine, heroin, sodium salicylate) tranquilizers, cholinergic drugs (e.g., acetylcholine, metacholilne), epinephrine and norepinephrine, dopamine, histamine, serotonin, glutathione, vitamin C, vitamin E, and hormones (e.g., estrogen).

Other radioprotective agents can include, but are not limited to cyanide, derivatives of nucleic acids (e.g., ATP), sodium fluoracetate, para-aminopropiophenone (PAPP), mellitin, endotoxins, imidazole, adenosine 3′,5′-cyclic monophosphate (cAMP), antibiotics, lipids (e.g. olive oil), erythropoietin, carbon monoxide (competes with hemoglobulin), hydrochloric mercaptoethylamine (MEA), sodium hydrogen S-(2-aminoethyl) phosphorothioic acid (WR-638), S-2-(3-aminopropylamino)ethyl phosphorothioic acid (WR-2721) S-2-(3-aminopropylamino) propylphosphorothioic acid (WR-44923), natural polyamines putrescine (1,4-Diaminobutane), spermidine and spermine. Other radioprotectors can include, but are not limited to nitroxide Tempol (4-hydroxy-2,2,6,6,-tetramethylpiperidine-1-oxyl), calcium antagonists (diltiazem, nifedipine and nimodipine), stobadine and bacterial endotoxins.

Immunomodulators are another class of radioprotectors that can enhance survival in irradiated animals. The most extensively studied cytokines regarding their radioprotective action are: interleukin-1 (IL-1), tumor necrosis factor alpha (TNF-.alpha.), granulocyte colony-stimulating factor (G-CSF) and granulocyte-macrophage CSF (GM-CSF). Another immunomodulator that is a radioprotective agent is AS101 (ammonium trichloro(dioxyethylene-O—O′) Tellurate) which stimulates the production of a variety of cytokines and presents radioprotective activity in mice.

As already mentioned hereinbefore, the selected tousled-like kinase activators that can be used in the context of the present invention include all substances that increase the stimulation or activation of a tousled-like kinase activity.

By increasing of stimulation or activation of tousled-like kinase is meant any increase in the activation of the kinase, which need only show a therapeutically significant activation of the kinase.

In one embodiment, activation of the receptor stimulation or activation, as defined by the present invention, means activation resulting from various interaction of the composition and the gene, transcript, receptor or its ligand. By interaction is meant sufficient physical or chemical interaction, such that tousled-like kinase activity is inhibited. One of skill in the art would appreciate that examples of such chemical interactions, which include association or bonding, are known in the art and include covalent bonding, ionic bonding, hydrogen bonding, etc.

In another embodiment in accordance with the present invention, the selected tousled-like kinase activator binds directly to the receptor. Additional tousled-like kinase activators can easily be determined using well-known methods.

Phenothiazines

Phenothiazines suitable for the compositions described herein include, piperadine and piperazine phenothiazines, e.g., chlorpromazine, promazine, triflupromazine, methotrimeprazine, mesoridazine, thioridazine, fluphenazine, perphenazine, flupentixol, prochlorperazine, trifluoperazine. promethazine, thioridazine, and acetophenazine. In some embodiments, the phenothiazine is prochlorperazine. In some instances, the compositions described herein comprise suitable derivatives of phenothiazines. In some embodiments, derivatives of phenothiazines allow for enhanced absorption across biological membranes. Suitable derivatives of phenothiazines include, e.g., lipid conjugates and/or prodrugs e.g., ester derivatives of phenothiazines. Additional derivatives include pharmaceutically acceptable salts, solvates, isomers, tautomers, metabolites, analogs, or prodrugs thereof.

Phenothiazines such as, for example, promethazine, prochlorperazine, thioproperazine, fluopromazine, perphenazine, are used in the treatment of tumors. In some instances, the treatment of tumors requires administration of multiple doses within a 24 hour period. For example, the current recommended dosage of prochlorperazine in the treatment of tumors is: Oral Dosage Tablets 5 mg or 10 mg tablet 3 or 4 times daily; Rectal Dose 25 mg twice daily; Intramuscular dose initial dose of 5 to 10 mg (1 to 2 mL) injected deeply into the upper outer quadrant of the buttock, with a repeat dose every 3 or 4 hours; Intravenous dose 21/2 to 10 mg (½ to 2 mL) by slow I.V. injection.

In certain embodiments, the dose of phenothiazine (e.g., prochlorperazine) for administration of a composition described herein is about 0.1 mg to 5 mg of phenothiazine. In certain embodiments, the dose of phenothiazine (e.g., prochlorperazine) for administration of a composition described herein is 0.1 mg, 1 mg, 2 mg, 5 mg, 10 mg, 25 mg, 50 mg, 100 mg or 200 mg of phenothiazine. In certain embodiments, the dose of phenothiazine for administration is 3 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, or 50 mg of phenothiazine. In certain embodiments, the dose of phenothiazine for administration is less than 2 mg, 5 mg, 7.5 mg, or 10 mg of phenothiazine.

The compositions described herein allow for reduced frequency of dose administration. In some instances, the frequency of administration of a dose of a phenothiazine (e.g., prochlorperazine) composition for administration described herein is once a day. In some instances, the frequency of administration of a dose of a phenothiazine (e.g., prochlorperazine) composition for administration described herein is twice a day. In some instances, the frequency of administration of a dose of a phenothiazine (e.g., prochlorperazine) composition for administration described herein is once every 48 hours, every 36 hours, every 24 hours, every 12 hours, every 10 hours, or every 8 hours.

In certain embodiments, the antipsychotic is selected from acetophenazine, alizapride, amisulpride, amoxapine, amperozide, aripiprazole, benperidol, benzquinamide, bromperidol, buramate, butaclamol, butaperazine, carphenazine, carpipramine, chlorpromazine, chlorprothixene, clocapramine, clomacran, clopenthixol, clospirazine, clothiapine, clozapine, cyamemazine, droperidol, flupenthixol, fluphenazine, fluspirilene, haloperidol, iloperidone, loxapine, melperone, mesoridazine, metofenazate, molindone, perphenazine, pimozide, prochlorperazine, promethazine, olanzapine, penfluridol, pericyazine, pipamerone, piperacetazine, pipotiazine, promazine, remoxipride, risperidone, sertindole, spiperone, sulpiride, thiothixene, thioridazine, trifluoperazine, trifluperidol, ziprasidone, zotepine, and zuclopenthixol.

In certain embodiments, the antipsychotic is a phenothiazine antipsychotic. In certain embodiments, the phenothiazine antipsychotic is selected from prochlorperazine, trifluoperazine, fluphenazine, promethazine, perphenazine, chlorpromazine, and thioridazine, mesoridazine, and acetophenazine. In certain embodiments, the antipsychotic is selected from prochlorperazine, trifluoperazine, fluphenazine, and perphenazine. In certain embodiments, the antipsychotic is prochlorperazine. In certain embodiments, prochlorperazine is administered by inhalation. In certain embodiments, the inhalation of prochlorperazine has no sustained effect on bronchoconstriction. In certain embodiments, two or more phenothiazine antipsychotics are combined.

In certain embodiments, the dose of phenothiazine antipsychotic for administration is about 0.1 mg to 5 mg of fluphenazine or trifluoperazine. In certain embodiments, the dose of phenothiazine antipsychotic for administration is 0.1 mg, 1 mg, 2 mg, 5 mg, 10 mg, 25 mg, 50 mg, 100 mg or 200 mg of fluphenazine or trifluoperazine. In certain embodiments, the dose of phenothiazine antipsychotic for administration is about 3 mg to 40 mg of chlorpromazine, thioridazine, or mesoridazine. In certain embodiments, the dose of phenothiazine antipsychotic is 3 mg, 5 mg, 7.5 mg, 10 mg, 20 mg, 30 mg, 50 mg or 100 mg of chlorpromazine, thioridazine, or mesoridazine. In certain embodiments, the dose of phenothiazine antipsychotic for administration is about 0.5 mg to 100 mg of prochlorperazine, perphenazine, acetophenazine, or promethazine. In certain embodiments, the dose of phenothiazine antipsychotic for administration is 0.5 mg, 1 mg, 5 mg, 10 mg, 20 mg, 40 mg, 60 mg, or 100 mg of prochlorperazine, perphenazine, acetophenazine, or promethazine. In certain embodiments, the dose of phenothiazine antipsychotic for intravenous administration is about 1 to 9 mg of prochlorperazine. In certain embodiments, the dose of phenothiazine antipsychotic for intravenous administration is about 1 to 5 mg of prochlorperazine.

In certain embodiments, the phenothiazine antipsychotic is prochlorperazine administered at a dosage of about 1 to 100 mg. In one example, 10 mg/mL of the phenothiazine antipsychotic trifluoperazine.

In certain embodiments, the antipsychotic is administered via any medically acceptable route of drug delivery. Exemplary nonlimiting routes of drug delivery include, but are not limited to, intranasally, intramuscularly, intravenously, orally, parenterally, transdermally, and rectally.

In certain embodiments, the antipsychotic is administered orally. Exemplary nonlimiting ways to accomplish oral administration of the antipsychotic include, but are not limited to, tablets, effervescent tablets, capsules, granulates, and powders. In certain embodiments, pharmacologically active ingredients are mixed with an inert solid diluent. Exemplary inert solid diluents include, but are not limited to, calcium carbonate, calcium phosphate and kaolin. In certain embodiments, the antipsychotic is provided in the form of soft gelatin capsules wherein the active ingredients are mixed with an oleaginous medium, e.g., but not limited to, liquid paraffin or olive oil. In certain embodiments, the antipsychotic is administered topically by mouth. Exemplary nonlimiting ways to accomplish topical administration include, but are not limited to, buccal tablets, sublingual tablets, drops, and lozenges.

In certain embodiments, the antipsychotic is administered by injection. Exemplary nonlimiting types of injection of the antipsychotic include, but are not limited to, intravenous injection, intramuscular injection, and subcutaneous injection, for example by bolus injection or continuous intravenous infusion. In certain embodiments, formulations for injection may be presented in unit dosage form, e.g., in ampoules or in multi-dose containers, with or without one or more added preservatives. In certain embodiments, formulations for injection can take such forms as suspensions, solutions, or emulsions in oily or aqueous vehicles, and may contain formulatory agents such as suspending, stabilizing, and/or dispersing agents. In certain embodiments, the active ingredient may be in powder form for dilution with a suitable vehicle, e.g., sterile pyrogen-free water, before use.

In certain embodiments, the antipsychotic may be formulated in rectal compositions such as suppositories or retention enemas, e.g., containing certain conventional suppository bases such as cocoa butter or other glyceride.

In certain embodiments, the antipsychotic is administered by inhalation. In certain embodiments, administration by inhalation results in rapid drug absorption without the need for injection. In certain embodiments, the administration by inhalation of the antipsychotic is performed by administration of a composition to a patient in aerosol form such that the patient inhales the composition by mouth or endotracheal tube in the pulmonary tract. In certain embodiments, administration by inhalation is accomplished using an inhalation delivery device. In certain embodiments, administration by inhalation is accomplished using Staccato Prochlorperazine for Inhalation. Non-limiting exemplary inhalation delivery devices include, but are not limited to, nebulizers, metered-dose inhalers, dry-powder inhalers or other inhalers known to those skilled in the art.

Nonlimiting exemplary inhalation devices are disclosed, e.g., in U.S. patent application Ser. No. 10/633,876 and U.S. Ser. No. 10/633,877, both filed on Aug. 4, 2003. Certain exemplary devices comprise a heat-conductive substrate onto which a film of antipsychotic is deposited. In certain embodiments, the surface area of the substrate is sufficient to yield a therapeutic dose of the antipsychotic aerosol when used by a subject. In certain embodiments, the desired dosage and selected antipsychotic film thickness dictate the minimum optimal substrate area in accord with the following relationship: film thickness (cm).times.antipsychotic density (g/cm3).times.substrate area (cm2)=dose (g). In certain embodiments, the calculated substrate area for a 5 mg dose of prochlorperazine is about 2.5 to 500 cm2, and the film thickness is about 0.1 to 20 μm.

In certain embodiments, the antipsychotic compound is delivered as an aerosol. In certain embodiments, the mass median aerodynamic diameter (MMAD) of the aerosol particles is less than about 5 μm. In certain embodiments, the MMAD of the aerosol particles is less than about 3 μm. In certain embodiments, the MMAD is within a range of about 1 to 5 μm.

In certain embodiments, the composition comprising the antipsychotic further comprises a diluent appropriate for human administration. In certain embodiments, the diluent is water, saline, ethanol, propylene glycol, glycerol, or mixtures thereof.

In certain embodiments, the antipsychotic is delivered as a single compound. In certain embodiments, more than one antipsychotic are used in a composition or are separately administered. In certain embodiments, the antipsychotic is used in a composition or separately administered with one or more additional compounds utilized in pain management. Nonlimiting exemplary compounds utilized in pain management include, but are not limited to, non-steroidal anti-inflammatory drugs, opioids, psychostimulants, barbiturates, benzodiazepines, and other compounds known to those skilled in the art.

In certain embodiments, the actual effective amount of antipsychotic for a particular patient can vary according to at least one of the specific antipsychotic or combination of antipsychotics being utilized; the particular composition formulated; the mode of administration; the age, weight, and condition of the patient; and the severity of the episode being treated.

In certain embodiments, the patient in need of treatment is an animal. In certain embodiments, the animal is a mammal. In certain embodiments, the patient in need of treatment is a human patient.

In certain embodiments, the antipsychotic is delivered by a route of administration that results in a therapeutic systemic concentration of the antipsychotic in the patient being obtained rapidly after initiation of administration of the antipsychotic to the patient. In certain embodiments, the therapeutic systemic concentration of the antipsychotic is obtained within 30 minutes of initiation of administration. In certain embodiments, the therapeutic systemic concentration of the antipsychotic is obtained within 15 minutes of initiation of administration. In certain embodiments, the therapeutic systemic concentration of the antipsychotic is obtained within 1 minute, 2 minutes, 3 minutes, 5 minutes, 10 minutes, 15 minutes, or 30 minutes of initiation of administration when the antipsychotic is prochlorperazine. In certain embodiments, the therapeutic systemic concentration of the antipsychotic is 20 ng/mL or less. In certain embodiments, the therapeutic systemic concentration is 1 ng/mL, 5 ng/mL, 10.0 ng/mL, 12.5 ng/mL, or 15 ng/mL of prochlorperazine, within 1 minute, 2 minutes, 3 minutes, 5 minutes, 10 minutes, 15 minutes, or 30 minutes of administration.

In certain embodiments, the methods provide for administration to a subject in need of such treatment of one or more doses of the antipsychotic. In certain embodiments, the first dose is about 0.5 mg to 18 mg of the antipsychotic. In certain embodiments, the first dose is 0.5 mg, 1 mg, 5 mg, 10 mg, 15 mg, 25 mg, or 50 mg of the antipsychotic. In certain embodiments, the one or more additional doses are about 1 mg to 50 mg of the antipsychotic. In certain embodiments, the one or more additional doses are 1-25 mg of the antipsychotic. In certain embodiments, the given interval of time is the amount of time it takes for the antipsychotic to approximately reach peak plasma concentration. In certain embodiments, the given interval of time is 20 minutes or less. In certain embodiments, the given interval of time is 1 minute, 5 minutes, 10 minutes, 20 minutes, 30 minutes, 60 minutes, or 120 minutes.

In certain embodiments, the antipsychotic is prochlorperazine. In certain embodiments, less than 6 mg of prochlorperazine is administered. In certain embodiments, the administration of the antipsychotic is via inhalation. In certain embodiments, the antipsychotic to be inhaled is a condensation aerosol comprising prochlorperazine.

Polyphenols

In another embodiment in accordance with the present invention, the selected tousled-like kinase activator is a polyphenol. In another embodiment in accordance with the present invention, the selected tousled-like kinase activator is a flavonoid or bioflavonoid.

In another embodiment, the flavonoid is selected from the group of the flavonols, flavonol o-glycosides, flavonol- or flavonol o-glycoside-containing extracts. In another embodiment, the flavonols are quercetin.

In another embodiment, the flavonol o-glycosides are flavonol 3-glycosides, such as rutin, rutin sulfate, alpha-glycosylrutin, tiliroside, troxerutin and/or isoquercetin.

In another embodiment, the flavonoid is selected from the group of quercetin, morin, naringenin and hesperetin, taxifolin, afzelin, quercitrin, myricitrin, genistein, apigenin and biochanin A, flavone, flavopiridol; the soy isoflavonoid, genistein; the tea catechin epigallocatechin gallate; flavonol, epicatechin, hesperetin, chrysin, diosmin, hesperidin, luteolin, and rutin.

In another embodiment, the bioactive polyphenol is at least one flavonoid. In another embodiment, the at least one flavonoid is selected from the group consisting of flavonones, flavones, dihydroflavonols, flavonols, flavandiols, leucoanthocyanidins, flavonol glycosodes, flavonone glycosides, isoflavonoids, and neoflavonoids. In another embodiment, the bioactive polyphenol is selected from the group consisting of quercetin, eriocitrin, neoeriocitrin, narirutin, naringin, hesperidin, hesperetin, neohesperidin, neoponcirin, poncirin, rutin, isorhoifolin, rhoifolin, diosmin, neodiosmin, sinensetin, nobiletin, tangeritin, catechin, catechin gallate, epigallocatechin, epigallocatechin gallate, anthocyanin, heptamethoxyflavone, curcumin, resveratrol, naringenin, tetramethoxyflavone, kaempferol, rhoifolin, and oolong tea polymerized polyphenol. In another embodiment, the polyphenol is selected from the group consisting of curcumin, rutin, resveratrol, naringenin, hesperedin, and tetramethoxyflavone.

In another embodiment, the bioflavonoids used for the preparation of the composition according to the invention may comprise at least one bioflavonoid, said bioflavonoid may be selected from the group consisting of flavonols, flavanones, flavones, flavan-3-ols, and anthocyanidins. In another embodiment in accordance with the present invention, the composition may comprise at least one bioflavonoid independently selected from the group consisting of quercetin, neoeriocitrin, naringin, and neohesperidin. In another embodiment in accordance with the present invention, the content of neoeriocitrin, naringin, neohesperidin may account for more than 40% of the total bioflavonoids in the composition such as more than 50%, for example more than 60%, such as more than 70%, for example more than 80%, such as more than 90% of the total bioflavonoids in the composition. In one preferred embodiment according to the invention neoeriocitrin, naringin, neohesperidin account (on per weight) for 85% of the total bioflavonoids in the composition (neoeriocitrin 9%, naringin 36%, neohesperidin 40%). The analysis of said extracted bioflavonoids is shown in example 13.

In one embodiment of the invention the at least one flavonoid is independently selected from the group consisting of the subgroup of flavonols, the subgroup of flavanones, the subgroup of flavones, the subgroup of flavan-3-ols, and the subgroup of anthocyanidins.

In another embodiment in accordance with the present invention, the selected tousled-like kinase activator is a flavonol compound.

In another embodiment in accordance with the present invention, the flavonol compound is a catechin compound selected from the group consisting of epicatechin, epigallocatechin gallate, gallocatechin, epicatechin gallate, epigallocatechin, and combinations and derivatives thereof.

In another embodiment in accordance with the present invention, the flavonol compound is a compound selected from the group consisting of 3-hydroxyflavone, azaleatin, fisetin, galangin, gossypetin, kaempferide, kaempferol, isorhamnetin, morin, myricetin, natsudaidain, pachypodol, quercetin, rhamnazin, rhamnetin, chrysin, apigenin, fisetin, kaempferol, luteolin, galangin, gossypetin, morin, myricetin, naringin, quercetin, robinetin, anthocyanins, rutin, hesperidin, taxifolin, catechinic acid, epicatechol, epicatechol gallate, gallocatechol, epigallocatechol gallate, tangeretin, eriodictyol, naringenin, rutin, troxerutin, (quercetinrutin), esculin, esculetin, skimmin, and umbelliferon.

In another embodiment, the selected tousled-like kinase activator is used in combination with an antioxidant co-agent suitable for systemic administration via the oral route is selected from the group consisting of alpha-tocopherol, beta-tocopherol, gamma-tocopherol, delta-tocopherol, epsilon-tocopherol, zeta1-tocopherol, zeta2-tocopherol, eta-tocopherol, citric acid, potassium citrate monohydrate, citric acid monohydrate, coenzyme Q.sub.n where n=1-12, L-selenocysteine, L-selenomethionine, butylated hydroxytoluene, butylated hydroxyanisole, propyl gallate, dodecylgallate, tert-butylhydroquinone, dihydrolipoic acid, prostaglandin B1 oligomers, 2-aminomethyl-4-tert-butyl-6-iodophenol, 2-aminomethyl-4-tert-butyl-6-propionylphenol, 2,6-di-tert-butyl-4-[2′-thenyl]-phenol, N,N′-diphenyl-p-phenylenediamine, ethoxyquin, probucol, ebselen, 5-[[3,5-bis(1,1-dimethylethyl)-4-hydroxyphenyl]-methylene]-3-(dimethylami-no)-4-thiazolidinone, 5-[[3,5-bis(1,1-dimethylethyl)-4-hydroxyphenyl]methylene]-3-(methylamino)-1-4-thiazolidinone, D-myoinositol-1.2.6-trisphosphate, nordihydro-guaiaretic acid, deferoxamine mesylate, tirilazad mesylate, derivative of tirilazad in which the steroid portion of the chemical structure has been replaced with the tetramethyl chroman portion of d-alpha tocopherol, trimetazidine, N,N′-dimethylthiourea, 2-(2-hydroxy-4-methylphenyl)aminothiazole hydrochloride, selenium, aspirin, potassium salicylate, calcium acetyl-salicylate, choline salicylate, imidazole salicylate, choline magnesium trisalicylate, magnesium salicylate, salsalate, parthenolide, daidzin, genistein, quercetin, morin, curcumin, apigenin, sesamol, chlorogenic acid, fisetin, ellagic acid, quillaia saponin, capsaicin, ginsenoside, silymarin, kaempferol, ginkgetin, bilobetin, isoginkgetin, isorhamnetin, herbimycin, rutin, bromelain, levendustin A and erbstatin.

Combination Therapies

Another aspect of this invention is directed towards a method of treating cancer in a subject in need thereof, comprising administration of a TLK modulating compound of this invention or a pharmaceutically acceptable salt thereof, and an additional therapeutic agent. In some embodiments, the method comprises the sequential or co-administration of the compound or a pharmaceutically acceptable salt thereof, and the additional therapeutic agent.

In some embodiments, the additional therapeutic agent is an anti-cancer agent. In other embodiments, the additional therapeutic agent is a DNA-damaging agent. In yet other embodiments, the additional therapeutic agent is selected from radiation therapy, chemotherapy, or other agents typically used in combination with radiation therapy or chemotherapy, such as radiosensitizers and chemosensitizers.

Examples of DNA-damaging agents that may be used in combination with compounds of this invention include, but are not limited to platinating agents, such as carboplatin, nedaplatin, satraplatin and other derivatives; topo I inhibitors, such as topotecan, irinotecan/sn38, rubitecan and other derivatives; antimetabolites, such as folic family (methotrexate, pemetrexed and relatives); purine antagonists and pyrimidine antagonists (thioguanine, fludarabine, cladribine, cytarabine, gemcitabine, 6-mercaptopurine, 5-fluorouracil (5fu) and relatives); alkylating agents, such as nitrogen mustards (cyclophosphamide, melphalan, chlorambucil, mechlorethamine, ifosfamide and relatives); nitrosoureas (eg carmustine); triazenes (dacarbazine, temozolomide); alkyl sulphonates (eg busulfan); procarbazine and aziridines; antibiotics, such as hydroxyurea, anthracyclines (doxorubicin, daunorubicin, epirubicin and other derivatives); anthracenediones (mitoxantrone and relatives); streptomyces family (bleomycin, mitomycin c, actinomycin); and ultraviolet light.

Other therapies or anticancer agents that may be used in combination with the inventive TLK modulating agents of the present invention include surgery, radiotherapy (in but a few examples, gamma-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, the dna damaging agents listed herein, spindle poisons (vinblastine, vincristine, vinorelbine, paclitaxel), podophyllotoxins (etoposide, irinotecan, topotecan), nitrosoureas (carmustine, lomustine), inorganic ions (cisplatin, carboplatin), enzymes (asparaginase), and hormones (tamoxifen, leuprolide, flutamide, and megestrol), gleevec, adriamycin, dexamethasone, and cyclophosphamide.

A TLK modulating compound of the instant invention may also be useful for treating cancer in combination with any of the following therapeutic agents: abarelix (plenaxis depot); aldesleukin (prokine); aldesleukin (proleukin); alemtuzumabb (campath); alitretinoin (panretin); allopurinol (zyloprim); altretamine (hexylen); amifostine (ethyol); anastrozole (arimidex); arsenic trioxide (trisenox); asparaginase (elspar); azacitidine (vidaza); bevacuzimab (avastin); bexarotene capsules (targretin); bexarotene gel (targretin); bleomycin (blenoxane); bortezomib (velcade); busulfan intravenous (busulfex); busulfan oral (myleran); calusterone (methosarb); capecitabine (xeloda); carboplatin (paraplatin); carmustine (bcnu, bicnu); carmustine (gliadel); carmustine with polifeprosan 20 implant (gliadel wafer); celecoxib (celebrex); cetuximab (erbitux); chlorambucil (leukeran); cisplatin (platinol); cladribine (leustatin, 2-cda); clofarabine (clolar); cyclophosphamide (cytoxan, neosar); cyclophosphamide (cytoxan injection); cyclophosphamide (cytoxan tablet); cytarabine (cytosar-u); cytarabine liposomal (depocyt); dacarbazine (dtic-dome); dactinomycin, actinomycin d (cosmegen); darbepoetin alfa (aranesp); daunorubicin liposomal (danuoxome); daunorubicin, daunomycin (daunorubicin); daunorubicin, daunomycin (cerubidine); denileukin diftitox (ontak); dexrazoxane (zinecard); docetaxel (taxotere); doxorubicin (adriamycin pfs); doxorubicin (adriamycin, rubex); doxorubicin (adriamycin pfs injection); doxorubicin liposomal (doxil); dromostanolone propionate (dromostanolone); dromostanolone propionate (masterone injection); elliott's b solution (elliott's b solution); epirubicin (ellence); epoetin alfa (epogen); erlotinib (tarceva); estramustine (emcyt); etoposide phosphate (etopophos); etoposide, vp-16 (vepesid); exemestane (aromasin); filgrastim (neupogen); floxuridine (intraarterial) (fudr); fludarabine (fludara); fluorouracil, 5-fu (adrucil); fulvestrant (faslodex); gefitinib (iressa); gemcitabine (gemzar); gemtuzumab ozogamicin (mylotarg); goserelin acetate (zoladex implant); goserelin acetate (zoladex); histrel in acetate (histrelin implant); hydroxyurea (hydrea); ibritumomab tiuxetan (zevalin); idarubicin (idamycin); ifosfamide (ifex); imatinib mesylate (gleevec); interferon alfa 2a (roferon a); interferon alfa-2b (intron a); irinotecan (camptosar); lenalidomide (revlimid); letrozole (femara); leucovorin (wellcovorin, leucovorin); leuprolide acetate (eligard); levamisole (ergamisol); lomustine, ccnu (ceebu); meclorethamine, nitrogen mustard (mustargen); megestrol acetate (megace); melphalan, 1-pam (alkeran); mercaptopurine, 6-mp (purinethol); mesna (mesnex); mesna (mesnex tabs); methotrexate (methotrexate); methoxsalen (uvadex); mitomycin c (mutamycin); mitotane (lysodren); mitoxantrone (novantrone); nandrolone phenpropionate (durabolin-50); nelarabine (arranon); nofetumomab (verluma); oprelvekin (neumega); oxaliplatin (eloxatin); paclitaxel (paxene); paclitaxel (taxol); paclitaxel protein-bound particles (abraxane); palifermin (kepivance); pamidronate (aredia); pegademase (adagen (pegademase bovine)); pegaspargase (oncaspar); pegfilgrastim (neulasta); pemetrexed disodium (alimta); pentostatin (nipent); pipobroman (vercyte); plicamycin, mithramycin (mithracin); porfimer sodium (photofrin); procarbazine (matulane); quinacrine (atabrine); rasburicase (elitek); rituximab (rituxan); sargramostim (leukine); sargramostim (prokine); sorafenib (nexavar); streptozocin (zanosar); sunitinib maleate (sutent); talc (sclerosol); tamoxifen (nolvadex); temozolomide (temodar); teniposide, vm-26 (vumon); testolactone (teslac); thioguanine, 6-tg (thioguanine); thiotepa (thioplex); topotecan (hycamtin); toremifene (fareston); tositumomab (bexxar); tositumomab/i-131 tositumomab (bexxar); trastuzumab (herceptin); tretinoin, atra (vesanoid); uracil mustard (uracil mustard capsules); valrubicin (valstar); vinblastine (velban); vincristine (oncovin); vinorelbine (navelbine); zoledronate (zometa) and vorinostat (zolinza).

For a comprehensive discussion of updated cancer therapies see, http://www.nci.nih.gov/, a list of the FDA approved oncology drugs at http://www.fda.gov/cder/cancer/druglistframe.htm, and The Merck Manual, Seventeenth Ed. 1999, the entire contents of which are hereby incorporated by reference.

Compositions for Administration into a Subject

The TLK kinase modulators or pharmaceutical salts thereof may be formulated into pharmaceutical compositions for administration to animals or humans. These pharmaceutical compositions, which comprise an amount of the TLK inhibitor effective to treat or prevent the diseases or conditions described herein and a pharmaceutically acceptable carrier, are another embodiment of the present invention.

The exact amount of compound required for treatment will vary from subject to subject, depending on the species, age, and general condition of the subject, the severity of the infection, the particular agent, its mode of administration, and the like. The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

In some embodiments, these compositions optionally further comprise one or more additional therapeutic agents. For example, chemotherapeutic agents or other anti-proliferative agents may be combined with the compounds of this invention to treat proliferative diseases and cancer. Examples of known agents with which these compositions can be combined are listed above under the “Combination Therapies” section and also throughout the specification. Some embodiments provide a simultaneous, separate or sequential use of a combined preparation.

Administering with Another Agent

Those additional agents may be administered separately, as part of a multiple dosage regimen, from the tousled-like kinase inhibitor-containing compound or composition.

Alternatively, those agents may be part of a single dosage form, mixed together with the tousled-like kinase inhibitor in a single composition.

Another aspect of this invention is directed towards a method of treating cancer in a subject in need thereof, comprising the sequential or co-administration of a compound of this invention or a pharmaceutically acceptable salt thereof, and an anti-cancer agent. In some embodiments, the anti-cancer agent is selected from platinating agents, such as cisplatin, oxaliplatin, carboplatin, nedaplatin, or satraplatin and other derivatives; topo I inhibitors, such as camptothecin, topotecan, irinotecan/sn38, rubitecan and other derivatives; antimetabolites, such as folic family (methotrexate, pemetrexed and relatives); purine family (thioguanine, fludarabine, cladribine, 6-mercaptopurine and relatives); pyrimidine family (cytarabine, gemcitabine, 5-fluorouracil and relatives); alkylating agents, such as nitrogen mustards (cyclophosphamide, melphalan, chlorambucil, mechlorethamine, ifosfamide, and relatives); nitrosoureas (e.g. carmustine); triazenes (dacarbazine, temozolomide); alkyl sulphonates (e.g. busulfan); procarbazine and aziridines; antibiotics, such as hydroxyurea; anthracyclines (doxorubicin, daunorubicin, epirubicin and other derivatives); anthracenediones (mitoxantrone and relatives); streptomyces family (bleomycin, mitomycin c, actinomycin) and ultraviolet light.

### Kit Embodiments

In certain embodiments, kits for use herein comprising an antipsychotic and an inhalation delivery device are provided. In certain embodiments, the antipsychotic is selected from acetophenazine, alizapride, amisulpride, amoxapine, amperozide, aripiprazole, benperidol, benzquinamide, bromperidol, buramate, butaclamol, butaperazine, carphenazine, carpipramine, chlorpromazine, chlorprothixene, clocapramine, clomacran, clopenthixol, clospirazine, clothiapine, clozapine, cyamemazine, droperidol, flupenthixol, fluphenazine, fluspirilene, haloperidol, iloperidone, loxapine, melperone, mesoridazine, metofenazate, molindone, perphenazine, pimozide, prochlorperazine, promethazine, olanzapine, penfluridol, pericyazine, pipamerone, piperacetazine, pipotiazine, promazine, remoxipride, risperidone, sertindole, spiperone, sulpiride, thiothixene, thioridazine, trifluoperazine, trifluperidol, ziprasidone, zotepine, and zuclopenthixol. In certain embodiments, the kits comprise a phenothiazine antipsychotic. In certain embodiments, the kits comprise a phenothiazine antipsychotic, which is selected from prochlorperazine, trifluorperazine, fluphenazine, promethazine, perphenazine, chlorpromazine, thioridazine, mesoridazine, and acetophenazine. In certain embodiments, the phenothiazine antipsychotic is about 1 to 18 mg prochlorperazine.

In certain embodiments, the kits comprise more than one dose of phenothiazine antipsychotic. In certain embodiments, the kits further comprise instructions for use. In certain embodiments, the kits comprise an inhalation delivery device, which produces a condensation aerosol.

### Example 1

TPH1^{-/-}, and corresponding wild type control animals (C57BL/6J genetic background)

Identification of TLK inhibitors: Inhibition of TLK1B autophosphorylation by phenothiazines

To identify inhibitors of TLK, we developed a high throughput screen with recombinant TLK1B, a small Rad9 peptide and the ADP Hunter reagent (DiscoveRx). Using this assay, the Innovative North Louisiana Experimental Therapeutics (INLET) screening core (affiliated with the FWCC) screened the Prestwick library, two other proprietary libraries, and a subset of the ChemDiv library (˜6000 compounds).

Following the initial fluorescent screen at a compound concentration of ˜5 μM, potential hits were tested with a more sensitive auto kinase assay with γ 32P ATP. Inhibition of auto phosphorylation was confirmed by TCA precipitable counts (FIG. 1A). The inhibitors are highly specific for TLKs. A commissioned KinomeScan (DiscoverRx—see link below for list: http://www.discoverx.com/services/drug discovery development services/kinase profiling/kinomescan) with THD revealed that no other kinase in the panel was significantly inhibited (>60% at 10 μM), and the compounds do not resemble ATP (the kinase assay dataset is available upon request for THD and PPH). FIG. 1B shows that the drugs worked specifically at low μM concentration after immunoprecipitation (IP) of TLK1 from 293T cells, and interestingly, the drugs remained associated with the protein, retaining their inhibition even after removal from the medium additional drug was not added to the IP in the kinase reaction. We have additional evidence for direct binding of these compounds to the recombinant protein (to be published elsewhere).

The TLK inhibitors delay DSB repair Kinetics of repair in episomes containing a single HO generated DSB, and slower dissolution of H2AX foci induced by Bleocin treatment.

We have used several assays to study DSB repair. In initial studies, the inhibitors delayed the repair kinetics of the single DSB introduced with Ad HO 4 on a novel multi copy episomal repair system (FIG. 2A). This system offers the advantage of generating 50 100 DSBs per cell that are all equivalent, genetically and in ends type, and in their chromatin context. The repair appears to be primarily accurate NHEJ (simple plasmid reclosure) for these episomes, as we have not yet obtained evidence of formation of concatomers, even when the population of plasmids was not completely cut, and thus offering intact template strands for HRR between plasmid molecules and presumably resulting in Holliday junctions and concatomeric units that we were unable to detect on Southern blots (work to be published elsewhere). Note also that this episomal system is fully capable of recapitulating the DDR induced by multiple genomic DSBs in mammalian cells, such as activation of ATM, Chk1 mediated inhibitory phosphorylation of TLK1 (S695), and generation γH2AX (FIG. 2B) most likely on the episomal chromatin itself, just as was reported for plasmids replicating in yeast.

The inhibitors also caused markedly increased sensitivity to radiation (IR) or Bleocin that could be explained by inhibition of DSB repair. This was shown as slower regression of DSB repair foci (γH2AX), which shows a representation of the main features we observed after DSB induction, namely that there were generally more foci per cell, they were brighter, and persisted for longer periods in presence of TLK inhibitors.

Repair of a DSB In Vitro

We have developed a system using nuclear extract of MM3MG cells that allows us to monitor repair of a plasmid cut with EcoRI, which leaves a cohesive 5′ overhang. Ligation of the ends and circularization of the plasmid, concomitant with chromatin formation, results in introduction of negative supercoils. Similar systems were described for stimulation of chromatin assembly by the histone chaperone CAF1 20. We showed that the addition of TLK1B hastened the repair and supercoiling of the plasmid with some involvement of Asf1; formation of chromatin in such assays is confirmed by analyses of regularly spaced nucleosomal ladders with MNase. While initial assays dealt with ligation of cohesive ends, we subsequent tested the ligation of incompatible ends (more representative of damaged ends typically obtained with XRT or RMT) that can be ligated only after some processing with a Rad9 dependency 5. Successful end joining and simultaneous plasmid supercoiling was monitored as an increased mobility (FIG. 4). Transformation of bacteria and analysis of the repaired junctions revealed a mix of fill in and resection of the ends 5. In new experiments here, a panel of TLK inhibitors reduced end filling and repair/religation with a Rad9 dependency (example in FIG. 4), indicating that the inhibitors also have a direct effect on DSB repair. Notably, 9 1 1 is known to recruit repair polymerases at incompatible ends to promote filling, which must precede ligation/supercoiling as only closed plasmid can be supercoiled.

Inhibition of Rad9 Phosphorylation

The inhibitors worked as expected on the pattern of phosphorylation of Rad9 S328. After treatment of DU145 cells with H2O2 for 10 min to generate breaks, there is a corresponding wave of phosphorylation and dephosphorylation of S328 after completion of repair (FIG. 5) that matches the pattern of activity of TLK1/1B TLKs are the only known kinases that phosphorylate Rad9 S328 4. Generation of DSBs causes transient inhibition of TLK1 and simultaneous synthesis of TLK1B, which then results in hyper phosphorylation of S328 upon restoration of kinase activity [FIG. 5A; 5]. Once H2O2 is degraded and most DNA breaks are repaired, the phosphorylation returns to baseline.

The inhibitors, PPH (LS125) in particular, blocked the activation of TLK and consequent phosphorylation of Rad9 (S328) (5C). This resulted in persistent activation of Chk1 (P Chk1 S345) (5D), which in turn resulted in prolonged phosphorylation of Rad17 (5E) and block in release of the RFC/Rad17/9 1 1 clamp loader/clamp complex at the repaired junctions (see model in Sup. FIG. 8), and previous results of ChIP analysis in cells expressing a KD TLK 5). This leads to a failure to deactivate the checkpoint and consequently increased cell death 5. In fact, cleavage of cdc25A, a target of Chk1 and key regulator of cdk2 and cdk4, is elicited much faster in the presence of LS125 (5F) further explaining the rapid and prolonged cell cycle arrest Similar experiments with doxo gave very similar results (Sup. FIG. 5). A key role of TLKs is then to participate in restoration of cell cycle upon completion of repair. Interfering with this process has dire consequences for cell viability; more likely for cancer cells that are genetically more unstable and already have defects in DNA repair functions, if not just for their reliance on these kinases.

Effects on Cell Cycle and Viability—RMT Potentiation In Vitro

The TLK inhibitors (e.g. TFP-LS392) even at 15 μM have little effect on the cell cycle or other deleterious effects, as shown by a very modest increase in (apoptotic) sub G1 cells, with very modest evidence of cleaved PARP (Sup. FIG. 2). In contrast, when combined with doxo, the inhibitors caused massive apoptosis and a partial S phase arrest, which coincidentally corresponds to the cell cycle phase when TLK activity is maximal6. A matching experimental set was processed by WB for evidence of cleaved PARP, and possible effects on ClnD1 accumulation, which occurs in late G1 and throughout S phases. Indeed concomitant treatment of doxo+LS392 caused maximal increase in ClnD1 levels, and corresponding with the apoptotic profile, PARP cleavage was highest in this treatment group (Sup FIG. 2, inset). The

inhibitors had generally similar potency in additive killing with doxo in both DU145 and PC3 (FIG. 6). The RMT potentiation is not limited to these two CaP cell lines, but is also true for MDA MB231 BCA cells (FIG. 6). For this experiment, we use MDA MB231 Luc cells, so that viability could be measured directly by luminescence, which is directly proportional to the number of cells and their health, and does not rely on viability dyes that can reach signal saturation—chemo luminescence in these cells is linear over several orders of magnitude. The TLK inhibitors by themselves had little effect on MDA231 viability and doubling (Sup. FIG. 3). The low toxicity of these TLK inhibitors is evident in the dose response where increasing the drug concentrations up to 15 μM had only modest effect on viability (FIG. 6), whereas a significant increase in cell killing was observed with increasing doxo or Bleomycin and the TLK inhibitors at 5 82 M (FIG. 6). This is consistent with their proposed mechanism as inhibitors of DSB repair. In a larger panel of cell lines, the TLK phenothiazine inhibitors did not affect doubling in most cells, but there was slight inhibition a few cell lines (Sup. FIG. 3).

Animal Studies

PC 3 model. A study was undertaken to investigate the in vivo efficacy of THD compared to doxo and to determine any sensitization conferred to the tumor cells by treatment with a combination of the two drugs. Eighty male SCID/bg mice received 1×106 PC 3 (human CaP cells) cells subcutaneous into their flanks. At day 49 post s inoculation, we began weekly tumor measurements by caliper [volume=(L×W2)/0.52)]. The PC 3 model in our hands resulted in slow growing tumors without the typical exponential phase that is optimal to support a case for growth inhibition in treated groups. There was no difference in tumor size between all groups (n=9 each) at the initiation of the treatments. At day 54, daily oral gavages of THD (0.25 mg in water) were given, and intraperitoneal injections of doxo (1 mg/kg; see arrows in FIG. 7) were at days 54, 68, and 95 (last day before euthanasia). In one arm of the study, 5 and 10 mg/kg doses of doxo (with and without THD) were administered, but these however published doses proved to be too toxic. There was statistically significant difference in tumor growth only at day 79 and day 86 for the treated groups (p<0.05 and 0.02, respectively). However, a definite trend was seen for doxo reducing tumor growth, at sub curative doses. Most interestingly, THD had a similar trend of tumor growth inhibition as doxo, a standard of chemotherapy, but without any toxicity or behavioral changes. In this experiment, the combination of THD and low doxo did not prove to be statistically more efficacious than either drug alone, although there could an “additive” trend that is more difficult to prove for statistical significance, but the fact that THD was already almost as effective as low doxo in controlling tumor progression was a main cause for the apparent lack of the synthetic effects that we instead observed for PC 3cells in culture. There is a curve inflection following the second dose of doxo for both groups (+/THD), and considering that the dose and schedule of doxo administration was not fully optimized, we concluded that perhaps a more quantitative model (MDA 231 Luc cells) to more precisely measure tumor growth could yield more definitive results. In any case, the tumors were excised at the end of the experiment (before the control group tumors reached the preset end point of 1500 mm3) and processed for IHC analysis. The mice were dosed on last day before euthanasia with doxo to induce bona fide DSBs.

IHC Results

Some features are immediately noticeable even upon H&E staining that could explain the tumor growth patterns. It was immediately obvious that, while treatment with doxo only slightly decreased the cellular density of the tumor, treatment with THD clearly reduced the tumor density, with fewer tumor cells and more fibrous interstitial tissue/stroma Staining for γH2AX revealed that few cells were positive in the untreated tumors, but a large proportion in mice treated with low doxo. Most importantly, there was a large proportion of positive staining cells also in tumors from mice treated with THD alone. We interpret this as being the result of accumulation of DNA damage, including DSBs, which are not promptly repaired, even as they may be expected to occur spontaneously in many cells in the harsh environment of tumor growth. This has in fact been reported before and was attributed to localized hypoxia 24. As anticipated, the combination of doxo+THD gave the highest density of γH2AX positive cells, and also the strongest signal per cell—indicative of larger numbers of unrepaired foci. Staining for PCNA revealed a pattern consistent with the previous observations. In controls, which showed the highest level of cellularity, a majority of the cells were clearly proliferative and stained strongly for PCNA. Tumors from mice treated with low doxo alone, showed a similar pattern and percentage of PCNA positive cells, but perhaps slightly lower cell density. In mice treated with THD alone, there were significantly fewer PCNA positive cells, consistent with the pattern of lesser cellularity. Nonetheless, the staining was intense in individual nuclei, indicating that the density of replication forks was not affected in those cells that were replicating, and hence, proliferation rate was probably normal. In contrast, the PCNA positive cells in tumors of mice treated with doxo+THD were not only fewer in number but were also was much lighter. This was observed in all slides we inspected for this group of mice. The staining was not negative, as one may think, since inspection at higher magnification confirmed the expected pattern of exclusively nuclear staining and lower intensity in nucleoli. It suggested instead a lower density of PCNA per nuclei, which would be expected from cells harboring stalled replication forks and replicating more slowly; and consistent with our observation, the aforementioned study on hypoxic tumor areas reported an inverse pattern of staining for Ki67 (very weak) where γH2AX positive cells were located 24. This is consistent with the γH2AX pattern, showing a large number of cells harboring repair foci, and thus likely arrested in replication (or progressing slowly through S phase). To further establish this, we looked at presence of RPA foci, which is indicative of stalled forks25. As expected from the large number of proliferating cells (PCNA positive) in control tumors, RPA1 Staining was very strong, and particularly in focal areas. RPA1 staining in tumors from doxo treated mice was interesting. The overall staining was weaker for most areas of the section, consistent with the idea we propose that cell proliferation is hampered by the need to repair DSBs. But interestingly, there were also areas of more intensely stained cells localized to necrotic spaces, which could be explained by the additional need for RPA during DNA repair in instances of damage that results in extensive formation of ssDNA 26, possibly in cells in the most hypoxic areas of the tumor. RPA staining in tumor from THD treated mice were similar to those treated with doxo, but in contrast, the staining in necrotic areas was weaker. In tumors from mice treated with doxo+THD, staining for RPA was very weak and consistent to that obtained for PCNA. We did not probe for presence of apoptotic cells (TUNEL), which are rarely found in vivo in any case. However, we did stain for P Rad9 (S328) to confirm that the effects were mediated via TLK inhibition, in what we propose is part of a checkpoint deactivation mechanism following repair of spontaneous DNA damage. Indeed, numerous cells stained lightly for P Rad9 in untreated tumor cells in fact, they are perhaps a larger proportion than those staining for γH2AX that presumably represent cells with unrepaired DSBs. Cells in tumors from animals treated with doxo showed much stronger staining for P Rad9, and perhaps the same density as those staining for γH2AX, suggesting that chemical induction of DSBs and repair occur concomitantly Treatment with

THD resulted in severe suppression of P Rad9, despite the clear evidence for presence of γH2AX in the same tumors (hence DSBs). Similarly, treatment with doxo+THD severely depressed P Rad9, which we ultimately interpret as incapacity to complete DSB repair and recovery from the DDR checkpoint.

MDA 231-Luc Model.

We used the breast cancer model cell line MDA 231-Luc, for more precise quantitation of tumor growth (photon emission is linear with the tumor cell number). Female SCID/bg mice (N=8 per group) received 1×10⁶ cells and when the tumors reached ˜500 mm3 (˜104 photons/sec) they were assigned to four treatment groups, as in the study above (Sup. FIG. 4). With this model, which showed typical exponential growth for the control group, THD alone was effective at controlling tumor increase, and actually was better than low doxo. In this experiment, a second course of doxo in the THD treated animals caused rapid and almost complete shrinkage of the tumors. In contrast, treatment with doxo alone was insufficient to stop growth of tumors, although there was a curve inflection at day 25 (10 days after doxo administration) that could be due to a delayed effect of doxo on either the tumors or the animals (not necessarily linked to accumulation of DSBs in tumor cells).

To determine how structurally related antipsychotic drugs might differ from each other and produce different biological effects, we analyzed optimized drug structures by quantum mechanics (QM) methods. Drugs of interest were built with MarvinSketch 5.3.2 (ChemAxon) for initial optimization of the 3D structure. For the panels shown here (Sup. FIG. 1), orbitals from the highest occupied molecular orbital (HOMO) and the lowest unoccupied molecular orbital (LUMO) have been depicted. We suggest that the binding site has very precise requirements, and that it may overlap with the ATP binding site. The crystal structure of the related kinase, ASK1, shows staurosporine bound at this site. The planar heterocyclic ring structure of staurosporine and its out of plane aminated glucose moiety are reminiscent of the structure of the phenothiazines. Bulky substituents on the main phenothiazine ring, such as fluorine groups, may only be tolerated in a certain orientation relative to the alkyl side chain, depending upon the reactivity of the side chain. Moreover, the fit at the binding site will be determined by whether active groups in the drugs (rings vs. side chains) serve as donors or acceptors in non bond interactions (e.g., it bonding and van der Waals interactions). While QM characterization of the drugs is not definitive, certain trends emerged from this analysis. In Sup. FIG. 1, three fourths of the phenothiazines with biological activity have a greater concentration of HOMO in the heterocyclic ring structure, whereas none of the four inactive compounds depicted do. When HOMO is localized in the alkyl substituent of the active drugs (e.g., TFP), it has the opposite phase of the inactive molecules (e.g., PCP). Based on these initial data, we suggest that the reactivity, or electron charge distribution, of the phenothiazine ring is an important determinant of the activity in TLK kinase assays. Conversely, greater reactivity of frontier orbitals in the alkyl substituents of the inactive drugs may promote interaction of these groups with TLK side chains that interferes with insertion of the phenothiazine ring into the narrow cleft at the ATP binding site. Because the precise features of the ATP binding site have been dramatically shaped by evolution, this site may be highly selective for drugs that only have slight differences in their 2 D structures. Together, this information may aid the design of more potent drugs in the future. Note also that our definition of “inactive compounds” is limited to concentrations <10 (space here) μM—we make no claims above that concentration, either in TLK inhibition or for other cellular effects, especially since undue toxicity is then observed. We should add that, the parental base compound, phenothiazine, is highly inhibitory, and so is the dye, methylene blue. Thus, the nitrogen alkyl substituents, which are critical for the dopaminergic effects, do not appear to matter for the inhibition of TLK, although they could partly explain the lack of it. In fact, methylene blue, which stains nucleic acids but not most proteins, remains so tightly bound to GST TLK1B that after its affixation to an Immobilon filter the color bound to the protein will not wash out.

While it is unclear whether the active phenothiazines bind an allosteric site or at/near the ATP pocket, we do know that

they do not prevent the binding of the Rad9 peptide, as we have done several experiments with a fluorescein tagged version of it. Another fact is clear: that the dopamine receptor (DR), in the unlikely case it was expressed in the cell lines we have studied here, plays little to no role in the effects we have reported. For instance, FF (inactive for TLK) is a more potent D2 receptor antagonist than either TFP (active) or THD (active), while PMZ does not have much activity as a D2 antagonist at all. Therefore, we find no obvious correlation between D2R antagonism and biological effects in our assays. This conclusion was also reached by Pam Silvers' group during a chemical genetic screen identifies inhibitors of regulated nuclear export of a Forkhead transcription factor in PTEN deficient tumor cells 28. They reported that a few phenothiazine antipsychotics blocked FOXOla export in 786 O cells and with similar potency (in contrast to our results on TLK activity). After initially raising the possibility that FOXO1a nuclear relocalization might be regulated by dopamine receptor signaling, they clearly demonstrated that this in fact was not the case, and instead implicated calmodulin as a possible target. We should unfortunately emphasize that their screens were carried out at 40 μM, and later none of their assays were carried out below 20 μM. We have already pointed out that at those concentrations these compounds are toxic to most mammalian cells, and hence any potential mechanistic conclusion on the redistribution of FOX1a export due to these compounds is dubious; and the implication of calmodulin suffers again from the fact that the protein requires concentrations >50 μM for inhibition in vitro, let alone in cells.

2. Discussion

Tousled Like Kinases and Rad9. The TLKs are highly conserved proteins in animal and vegetal kingdoms, with dual chaperone kinase functions that affect several process of chromatin assembly (rev. in 3). Only a few direct “interacting” substrates of TLKs have been identified, the principals being the histone chaperone Asf1, histone H3, and Rad9. This suggested that TLKs function in processes of chromatin assembly that have been identified so far in transcription; DNA repair; replication; and mitotic condensation of chromosomes. The human homolog, TLK1B, has invoked interest because of its established role in cell survival after DNA damage. In mammals, the primary TLK1 transcript is alternatively spliced in two main isoforms, TLK1 and TLK1B 36; TLK1B is subject to translational regulation and its synthesis is induced by DNA damage. Elevated expression of TLK1B promotes cell survival after radiation (IR) or doxo by facilitating DNA repair 19, while expression of a kinase dead mutant renders mammalian cells sensitive to IR 34. Evidence also exists for a link between TLKs and a DNA damage relay 7 that leads to transient kinase inhibition mediated by ATM via Chk1 by direct phosphorylation of TLK1 at S695 33. These findings identified a functional cooperation between ATM and Chk1 in propagation of a checkpoint response mediated by transient inhibition of TLK1, which may regulate processes involved in chromatin remodeling after damage 7, as well as assembly of repair components at damage sites and other aspects of DDR and recovery. Rad9, Rad1, and Hus1 form a trimeric complex (termed 9 1 1) that is structurally similar to the PCNA “sliding clamp”, which encircles the DNA conferring processivity to polymerases, and performing many other functions, including mediation of the checkpoint. 9 1 1 assembles at sites of damage via the genotoxin activated RFC Rad17 “clamp loader” 39. The 9 1 1 complex may then serve as a scaffold for assembly of DNA repair proteins, as well as engaging components for the DDR. We showed that TLK1B uniquely phosphorylates Rad9 at S328, and that this appears to play a key role in resumption of the cell cycle. However, TLK1B also had a function as a chaperone for Rad9 assembly at DSBs that was independent of its kinase function 4. We proposed that the regulated binding of 9 1 1 and TLK1B to DSBs recruits repair enzymes and a chromatin disassembly apparatus to promote efficient repair, and only subsequently TLKs participate in DDR disengagement and deactivation of the checkpoint 5, for which we have now presented additional evidence. A model for the role of S328 phosphorylation in release of 9 1 1 and consequent deactivation of the checkpoint is included (Sup. FIG. 6). Recent studies showcase the importance of the ATM dependent phosphorylation of Rad9 (S272) in maintenance of genomic stability 40.

Here we show for the first time a critical role for the phosphorylation of S328 by TLKs in checkpoint recovery from the DDR, and possibly directly in ends repair in vitro with a plasmid system (FIG. 4). This extends our previous studies with ES rad9/reconstituted with hRad9 (S328A) 4, and the use of TLK1B KD expressing cells engineered to contain a single HO mediated DSB 5. Since Rad9 is a critical mediator of the DDR checkpoint, and in repair (specifically of DSBs), we proposed that the TLK1 Rad9 interaction would be very important in implementing the mechanism of TLK1B mediated radioprotection 11. The significance of the TLK/Rad9 axis is clear for prostate cancer for which several studies have implicated Rad9's critical role in disease progression and prognosis. Nonetheless, elevated Rad9 expression, and some of its phosphorylated forms, has been noted also in BCA and correlated to prognosis.

TLKs are Targets for Therapeutic Intervention

The TLKs are becoming the center of much attention for their role in DSB repair and their potential contribution to cancers refractory to XRT or RMT, including cholangiocarcinomas, BCA and CaP. Note however, that probably most technologies directed at globally reducing the expression of TLKs are unlikely to result in effective and safe therapies. Silencing RNA mediated suppression of TLKs eventually kills all cells (normal or cancer) even without radiation or RMT. These are essential proteins as chaperones, while the kinase activity seems less critical, and needed for more complex and specialized functions, as shown here. In fact, we could produce a stable cell line of normal mammary fibroblasts expressing a TLK1 KD mutant (in presence of the wt endogenous TLKs), but we also used siRNA to complement the work, which arrested the cells and then killed them 34. Since the mechanism of action of TLKs in resistance to genotoxins via DNA repair is now fairly well elucidated 3, it would seem that adding TLK inhibitors could significantly improve standard therapy. While our screen for inhibitors of TLK is still ongoing, the fact that the first attempt has identified a panel of phenothiazine antipsychotics with >30 years of clinical use, immediately dictated a point to stop and take stock. And indeed we further looked at the full panel of commonly used phenothiazine antipsychotics, and only a subset had inhibitory activity on TLK and DSB repair in vitro and in cells. Hence, while this is not the first report that some of these compounds enhance cell killing with XRT or RMT via inhibition of DSB repair, the proposed generalization that all these drugs can do so is incorrect. Moreover, none of the previous studies identified the correct molecular/cellular target(s) for previously noted reasons in regard to effective doses. We can clearly exclude the involvement of DNA PK for several reasons. First, at sub toxic concentrations of TFP (10-20 μM) that completely inhibit TLKs, DNA PK is not inhibited reported inhibition in vitro: 0.1-0.2 mM. Second, the phenothiazine concentrations used in that study induced strong apoptotic and proteolytic conditions in cells resulting in significant degradation of DNA PK, and it was not even clear if the reported inhibition was for activity or from loss of intact protein. Lastly, we have additional results showing that TLKs and Rad9 (S328) phosphorylation are involved in restart of stalled replication forks after release from hydroxyurea (HU), and that TFP greatly extends this process with a delay of its linked phosphorylation of Rad9 (S328) after DDR deactivation. In contrast, DNA PK is not known to be involved in replication restart and is rather specific for NHEJ. While identifying the molecular target may not seem that important in terms of their potential use as adjuvants in clinical trials, in reality the best way to design an effective study is to have available intermediate biomarkers, like P Rad9 that can also be measured in blood lymphocytes, to establish the individual doses. So knowing the mechanism/target of the therapy is important. While we were completing our studies, a paper was published that reported the identification of THD during a screen to find compounds that selectively target Cancer Stem Cells 46, therefore adding another potential anti cancer target to the use of THD. More specifically, the authors used a presumed distinction between normal pluripotent cells (hPSC) and CSC, based on Oct4 GFP expression, to find compounds that induce differentiation to overcome neoplastic self renewal. Assuming that this is conceptually applicable to most cancers many of us think that most cancer cells are “stem cells” by at least the basic definition of unlimited self renewal their further conclusions that “these results suggest that dopamine receptors may serve as a biomarker for diverse malignancies” are not subsequently supported in their study. Certainly, we have not found very many cancer cell lines that were sensitive to THD for growth at the concentrations we used (Sup FIG. 3). All those cell lines were derived from patients, and there are very few reports of DR expression in cancer lines, like the small lung cancer cell line NCI H69.

Cancer and Schizophrenia

Since these drugs are used for treatment of severe mental illness, we looked at literature on the risk of cancer among individuals with schizophrenia. Surprisingly, despite the fact that patients with schizophrenia should have high comorbidity factors that should increase the cancer risk, it is a known paradox in the field that the risk of developing cancer is generally lower than what might be expected for both male and female common cancers. Below we discuss the case of BCA in individuals with schizophrenia, where more current information from larger studies is available, and we propose a possible explanation for these unexpected epidemiologic observations 51. A recent preclinical trial at Dana Farber was designed to establish doses of an oral PARP inhibitor for breast cancer prevention in BRCA mutation carriers. The underlying hypothesis is that tumor BRCA deficient tumor cells must rely upon alternative DNA repair pathways to maintain sub lethal levels of DNA damage, and that PARP inhibitors block these back up DNA repair pathways, promoting accumulation of lethal levels of DNA damage but spare normal cells that retain BRCA activity. The epidemiologic evidence supports a similar scenario with TFP as an inhibitor of TLKs and hence DNA repair in individuals with schizophrenia, possibly providing similar chemoprevention activity as a PARP inhibitor. An alternative explanation is that elevated TLK1B or TLK2 expression is a critical transition during development or progression of many BCAs, and preemptive inhibition of these kinases by phenothiazines could choke a key step in the formation of breast tumors.

TLKs, Schizophrenia, and Breast Cancer

Some phenothiazine antipsychotics (specifically: TFP, THD, PPH, PMZ, TEZ) that have been used to treat patients since the 1950s have been identified as TLK inhibitors. The link between schizophrenia therapy and TLK inhibition is an interesting possibility because, if the TLK inhibiting properties of these drugs are effective against BCA, then one would expect that women prescribed these drugs might display (a) a lower incidence of breast cancer compared to those who did not take the drugs and/or (b) an improved outcome after BCA diagnosis. While women with schizophrenia have multiple risk factors for breast cancer that might lead to expectations of substantially increased risk (smoking, obesity, low exercise, poor diet, treatment induced hyperprolactinemia), results of a systematic review of such studies 17 revealed that the incidence is either decreased, similar or only slightly increased from that of the general population.

Various hypotheses have been proposed for such findings, including protective genetic effects, anti tumor properties of neuroleptics.

Schizophrenia and Prostate Cancer

An analysis from 5 independent studies of CaP incidence in individuals with schizophrenia revealed in each case a significant decrease in incidence ranging from 0.49 to 0.76 49. Proposed explanations for this were: specific genetic factors; antipsychotic drug effects, either by being cancer protective or decreasing testosterone, or both; and lifestyle differences, such as prolonged hospitalization resulting in a decreased opportunity for heterosexual intercourse.

TLK1 In Vitro Kinase Assays

TlK1 immune complexes isolated from 200 g protein extracts using abtiserum made in our lab and Protein A beads. Adsorbed proteins were washed twice in kinase assay buffer (KAB) (50 mM HEPES pH 7.2, 10 mM MgCl₂, 5 mM MnCl₂, 2.5 mM EGTA, 1 mM DTT, 1 mM NaF, 0.2 mM sodium o vanadate, 2.5 g/ml leupeptin, 2 g/ml aprotinin) and incubated for 15-30 min at 30° C. in KAB supplemented with 10 M ATP, 10 Ci [−32P]ATP (>5000 Ci/mmol, GE). The proteins were separated on 10% SDS-PAGE gels, transferred to nitrocellulose before exposure on a Phosphorlmager; and then processed for WB with TLK1 antiserum. For kinase assays with recombinant TLK1B protein, the reactions were similar, and carried out with 10 ng TLK1B and 50 ng Rad9 peptide substrate. These were carried out to confirm the initial “hits” from the screen and further to obtain accurate ID50 values for the various inhibitors.

Plasmid In Vitro Repair Reactions

Preparation of Nuclear extract and plasmid religation coupled to chromatin assembly are described in 5. Ligation of a blunt end generated by EcoRV and one generated by EcoRI can only be repaired by the generation of a flush end at the EcoRI side, which could either take place by fill in of the EcoRI overhang and resection of the remaining 5′ ApA overhang or by fill-in with residual dTTP present in the extract. Where indicated, recombinant hRad9 was added at 100 nM final concentration.

Quantum Mechanics Molecular Mechanics (QM MM) Calculations

The various phenothiazine drugs were initially minimized with the MMFF forcefield in Discovery Studio 3.1 (Accelrys, San Diego, Calif.). Geometry optimization and molecular orbital visualization were then achieved using QM MM methods. Molecules were typed with the CHARM forcefield with MMFF94 partial charge settings. Density functional theory (DFT) methods with the VWN BP gradient corrected functional were used to calculate QM properties of the molecules. The DN DMo13 basis set was used with self consistent field (SCF) convergence at 1×10 4. The frontier orbitals—highest occupied molecular orbital (HOMO) and lowest unoccupied molecular orbital (LUMO) —were then visualized to compare the active vs. inactive drugs.

### REFERENCES

1. Davar D, Beumer J H, Hamieh L, Tawbi H. Role of PARP inhibitors in cancer biology and therapy. Curr Med Chem 2012; 19(23): 3907 [symbol] 21.
2. Lord C J, Garrett M D, Ashworth A. Targeting the double [symbol] strand DNA break repair pathway as a therapeutic strategy. Clin Cancer Res 2006; 12(15): 4463 [symbol] 8.
3. De Benedetti A. The Tousled [symbol] Like [symbol] Kinases as guardians of genome integrity. ISRN Molecular Biology 2012; 2012.
4. Sunavala [symbol] Dossabhoy G, De Benedetti A. Tousled homolog, TLK1, binds and phosphorylates Rad9; tlk1 acts as a molecular chaperone in DNA repair. DNA Repair 2009; 8: 87 [symbol] 102.
5. Canfield C, Rains J, De Benedetti A. TLK1B promotes repair of DSBs via its interaction with Rad9 and Asf1. BMC Mol Biol 2009; 10: 110.
6. Sillje H, Nigg E. Identification of human Asf1 chromatin assembly factors as substrates of Tousled [symbol] like kinases Curr Biol. 2001; 11(13): 1068 [symbol] 73.
7. Groth A, Lukas J, Nigg E, et al. Human Tousled like kinases are targeted by an ATM [symbol] and Chk1 [symbol] dependent DNA damage checkpoint. EMBO J. 2003; 22(7): 1676 [symbol] 87.
8. Byrnes K, De Benedetti A, Holm N, et al. Correlation of TLK1B in Elevation and Recurrence in Doxorubicin [symbol] Treated Breast Cancer Patients with High eIF4E Overexpression. J Am Coll Surg. 2007; 204(5): 925 [symbol] 33.
9. Stevens K N, Wang X, Fredericksen Z, et al. Evaluation of associations between common variation in mitotic regulatory pathways and risk of overall and high grade breast cancer. Breast Cancer Res Treat 2011.
10. Kelemen L E, Wang X, Fredericksen Z S, et al. Genetic variation in the chromosome 17q23 amplicon and breast cancer risk. Cancer Epidemiol Biomarkers Prey 2009; 18(6): 1864 [symbol] 8.
11. Ronald S, Sunavala [symbol] Dossabhoy G, Adams L, Williams B, De Benedetti A. The expression of Tousled kinases in CaP cell lines and its relation to radiation response and DSB repair. Prostate 2011; 71(13): 1367 [symbol] 73.
12. Polischouk A G, Holgersson A, Zong D, et al. The antipsychotic drug trifluoperazine inhibits DNA repair and sensitizes non small cell lung carcinoma cells to DNA double [symbol] strand break induced cell death. Mol Cancer Ther 2007; 6(8): 2303 [symbol] 9.
13. Gangopadhyay S, Karmakar P, Dasgupta U, Chakraborty A. Trifluoperazine stimulates ionizing radiation induced cell killing through inhibition of DNA repair. Mutat Res 2007; 633(2): 117 [symbol] 25.
14. Zong D, Haag P, Yakymovych I, Lewensohn R, Viktorsson K. Chemosensitization by phenothiazines in human lung cancer cells: impaired resolution of gammaH2AX and increased oxidative stress elicit apoptosis associated with lysosomal expansion and intense vacuolation. Cell Death Dis 2011; 2: e181.
15. Eriksson A, Yachnin J, Lewensohn R, Nilsson A. DNA [symbol] dependent protein kinase is inhibited by trifluoperazine. Biochemical and biophysical research communications 2001; 283(4): 726 [symbol] 61.
16. Perez R P, Handel L M, Hamilton T C. Potentiation of cisplatin cytotoxicity in human ovarian carcinoma cell lines by trifluoperazine, a calmodulin inhibitor. Gynecol Oncol 1992; 46(1): 82 [symbol] 7.
17. Hodgson R, Wildgust H J, Bushe C J. Cancer and schizophrenia: is there a paradox? J Psychopharmacol 2010; 24(4 Suppl): 51 [symbol] 60.
18. Fink M, Imholz D, Thoma F. Contribution of the serine 129 of histone H2A to chromatin structure. Mol Cell Biol 2007; 27(10): 3589 [symbol] 600.
19. Sunavala [symbol] Dossabhoy G, Balakrishnan S, Sen S, Nuthalapaty S, De Benedetti A. The radioresistance kinase TLK1B protects the cells by promoting repair of double strand breaks. BMC Mol. Biol. 2005; 6: 19.
20. Hoek M, Myers M P, Stillman B. An analysis of CAF [symbol] 1 [symbol] interacting proteins reveals dynamic and direct interactions with the KU complex and 14 [symbol] 3 [symbol] 3 proteins. J Biol Chem 2011; 286(12): 10876 [symbol] 87.
21. De Benedetti A. Tousled kinase TLK1B counteracts the effect of Asf1 in inhibition of histone H3 [symbol] H4-14 tetramer formation. BMC Res Notes 2009; 2: 128.
22. De Benedetti A. Tousled kinase TLK1B mediates chromatin assembly in conjunction with Asf1 regardless of its kinase activity. BMC Res Notes 2010; 3: 68.
23. Sukhanova M, D'Herin C, van der Kemp P, Koval V, Boiteux S, Lavrik O. Ddc1 checkpoint protein and DNA polymerase epsilon interact with nick [symbol] containing DNA repair intermediate in cell free extracts of Saccharomyces cerevisiae. DNA Repair (epub January 11) 2011.
24. Sun J D, Liu Q, Wang J, et al. Selective Tumor Hypoxia Targeting by Hypoxia [symbol] Activated Prodrug TH [symbol] 302 Inhibits Tumor Growth in Preclinical Models of Cancer. Clinical Cancer Research 2012; 18(3): 758 [symbol] 70.
Zhang J, Brown R P, Shaw M, et al. Immunolocalization of Kim [symbol] 1, RPA [symbol] 1, and RPA [symbol] 2 in Kidney of Gentamicin [symbol] , Mercury [symbol] , or Chromium [symbol] Treated Rats: Relationship to Renal Distributions of iNOS and Nitrotyrosine. Toxicologic Pathology 2008; 36(3): 397 [symbol] 409.
26. Robison J G, Lu L, Dixon K, Bissler J J. DNA Lesion [symbol] specific Co [symbol] localization of the Mrell/Rad50/Nbs1 (MRN) Complex and Replication Protein A (RPA) to Repair Foci. Journal of Biological Chemistry 2005; 280(13): 12927 [symbol] 34.
27. Bunkoczi G, Salah E, Filippakopoulos P, et al. Structural and functional characterization of the human protein kinase ASK1. Structure 2007; 15(10): 1215 [symbol] 26.
28. Kau T R, Schroeder F, Ramaswamy S, et al. A chemical genetic screen identifies inhibitors of regulated nuclear export of a Forkhead transcription factor in PTEN [symbol] deficient tumor cells. Cancer Cell 2003; 4(6): 463 [symbol] 76.
29. Carrera P, Moshkin Y, Gronke S, et al. Tousled [symbol] like kinase functions with the chromatin assembly pathway regulating nuclear divisions. Genes & Dev. 2003; 17(20): 2578 [symbol] 90.
30. Han Z, Saam J, Adams H, Mango S, Schumacher J. The C. elegans Tousled [symbol] like kinase (TLK [symbol] 1) has an essential role in transcription. Current Biology 2003; 13: 1921 [symbol] 29.
31. Wang Y, Liu J, Xia R, et al. The protein kinase TOUSLED is required for maintenance of transcriptional gene silencing in Arabidopsis. EMBO Rep 2007; 8(1): 77 [symbol] 83.
32. Sen S, De Benedetti A. TLK1B promotes repair of UV [symbol] damaged DNA through chromatin remodeling by Asf1. BMC Mol. Biol. 2006; 7: 37.
33. Krause D, Jonnalagadda J, Gatei M, et al. Suppression of Tousled [symbol] like kinase activity after DNA damage or replication block requires ATM, NBS 1 and Chk1. Oncogene 2003; 22(38): 5927 [symbol] 37.
34. Sunavala [symbol] Dossabhoy G, Li Y, Williams B, De Benedetti A. A dominant negative mutant of TLK1 causes chromosome missegregation and aneuploidy in normal breast epithelial cells. BMC Cell Biol. 2003; 4: 16.
35. Han Z, Riefler G M, Saam J R, Mango S E, Schumacher J M. The C. elegans Tousled [symbol] like kinase contributes to chromosome segregation as a substrate and regulator of the Aurora B kinase. Curr Biol 2005; 15(10): 894 [symbol] 904.
36. Li Y, DeFatta R, Anthony C, Sunavala G, De Benedetti A. A translationally regulated Tousled kinase phosphorylates histone H3 and confers radioresistance when overexpressed. Oncogene 2001; 20(6): 726 [symbol] 38.
37. Roos [symbol] Mattjus P, Hopkins K, Oestreich A, et al. Phosphorylation of Human Rad9 Is Required for Genotoxin [symbol] activated Checkpoint Signaling. J. Biol. Chem. 2003; 278(27): 24428 [symbol] 37.
38. Lieberman H B, Bernstock J D, Broustas C G, Hopkins K M, Leloup C, Zhu A. The role of RADS in tumorigenesis. J Mol Cell Biol 2011; 3(1): 39 [symbol] 43.
39. Lindsey [symbol] Boltz L, Bermudez V, Hurwitz J, Sancar A. Purification and characterization of human DNA damage checkpoint Rad complexes. PNAS 2001; 98(20): 11236 [symbol] 41.
40. Shin M H, Yuan M, Zhang H, Margolick J B, Kai M. ATM [symbol] dependent phosphorylation of the checkpoint clamp regulates repair pathways and maintains genomic stability. Cell Cycle 2012; 11(9).
41. Zhu A, Zhang C, Lieberman H. Rad9 has a functional role in human prostate carcinogenesis. Cancer Res. 2008; 68(5): 1267 [symbol] 74.
42. Hsu C, Chen Y, Ting H, et al. Androgen receptor (AR) NH2 [symbol] and COOH [symbol] terminal interactions result in the differential influences on the AR [symbol] mediated transactivation and cell growth. Mol. Endocrinol. 2005; 19: 350 [symbol] 61.
43. Chan V, Khoo U S, Wong M S, et al. Localization of hRad9 in breast cancer. BMC Cancer 2008; 8: 196.
44. Palaniyandi S, Odaka, Y, Green, W, Abreo, F, Caldito, G, De Benedetti, A, Sunavala [symbol] Dossabhoy, G Adenoviral Delivery of Tousled Kinase for the Protection Salivary Glands against Ionizing Radiation Damage. Gene Therapy 2011; 18: 275 [symbol] 82.
45. Takayama Y, Kokuryo T, Yokoyama Y, et al. Silencing of Tousled [symbol] like kinase 1 sensitizes cholangiocarcinoma cells to cisplatin-induced apoptosis. Cancer Letters 2010; 296: 27 [symbol] 34.
46. Sachlos E, Risueno R M, Laronde S, et al. Identification of Drugs Including a Dopamine Receptor Antagonist that Selectively Target Cancer Stem Cells. Cell 2012; 149: 1284 [symbol] 97.
47. Sangodkar J, Dhawan N S, Melville H, et al. Targeting the FOXO1/KLF6 axis regulates EGFR signaling and treatment response. J Clin Invest 2012; 122(7): 2637 [symbol] 51.
48. Chou F H, Tsai K Y, Su C Y, Lee C C. The incidence and relative risk factors for developing cancer among patients with schizophrenia: a nine [symbol] year follow [symbol] up study. Schizophr Res 2011; 129(23): 97 [symbol] 103.
49. Torrey E R Prostate cancer and schizophrenia. Urology 2006; 68(6): 1280 [symbol] 3.
50. Ji J, Sundquist K, Ning Y, Kendler K S, Sundquist J, Chen X. Incidence of Cancer in Patients With Schizophrenia and Their First [symbol] Degree Relatives: A Population [symbol] Based Study in Sweden. Schizophr Bull 2012.
51. Barak Y, Levy T, Achiron A, Aizenberg D. Breast cancer in women suffering from serious mental illness. Schizophr. Res. 2008; 102(1 [symbol] 3): 249 [symbol] 53.
52. Catts V S, Catts S V. Apoptosis and schizophrenia: is the tumour suppressor gene, p53, a candidate susceptibility gene? Schizophr Res 2000; 41(3): 405 [symbol] 15.
53. Cui D H, Jiang K D, Jiang S D, Xu Y F, Yao H. The tumor suppressor adenomatous polyposis coli gene is associated with susceptibility to schizophrenia. Mol Psychiatry 2005; 10(7): 669 [symbol] 77.
54. Park J K, Lee H J, Kim J W, et al. Differences in p53 gene polymorphisms between Korean schizophrenia and lung cancer patients. Schizophr Res 2004; 67(1): 71 [symbol] 4.
55. Yang Y, Xiao Z, Chen W, et al. Tumor suppressor gene TP53 is genetically associated with schizophrenia in the Chinese population. Neurosci Lett 2004; 369(2): 126 [symbol] 31.
56. Carrillo J A, Benitez J. Are antipsychotic drugs potentially chemopreventive agents for cancer? Eur J Clin Pharmacol 1999; 55(6): 487 [symbol] 8.
57. Strobl J S, Peterson V A. Tamoxifen [symbol] resistant human breast cancer cell growth: inhibition by thioridazine, pimozide and the calmodulin antagonist, W [symbol] 13. J Pharmacol Exp Ther 1992; 263(1): 186 [symbol] 93.
58. Motohashi N, Kawase M, Saito S, Sakagami H Antitumor potential and possible targets of phenothiazine [symbol] related compounds. Curr Drug Targets 2000; 1(3): 237 [symbol] 45.
59. Mazars A, Fernandez [symbol] Vidal A, Mondesert O, et al. A caspase [symbol] dependent cleavage of CDC25A generates an active fragment activating cyclin-independent kinase 2 during apoptosis. Cell Death Differ 2009; 16(2): 208 [symbol] 18.

All publications, patents and patent applications cited herein, whether supra or infra, are hereby incorporated by reference in their entirety to the same extent as if each individual publication, patent or patent application was specifically and individually indicated as incorporated by reference. It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein, will only be incorporated to the extent that no conflict arises between that incorporated material and the existing disclosure material.

It must be noted that, as used in this specification and the appended claims, the singular forms “a,” “an” and “the” include plural referents unless the content clearly dictates otherwise. Thus, for example, reference to a “colorant agent” includes two or more such agents.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

While it is apparent that the illustrative embodiments of the invention herein disclosed fulfill the objectives stated above, it will be appreciated that numerous modifications and other embodiments may be devised by one of ordinary skill in the art. Accordingly, it will be understood that the appended claims are intended to cover all such modifications and embodiments, which come within the spirit and scope of the present invention.

It should be noted that, when employed in the present disclosure, the terms “comprises,” “comprising,” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

What is claimed is:

1. A method of treating, managing or preventing a specific cancer, which comprises administering to a patient in need of such treatment, management or prevention a therapeutically or prophylactically effective amount of a selective tousled-like kinase inhibitory drug, or a pharmaceutically acceptable salt, solvate, or stereoisomer thereof.

2. A method of treating, managing or preventing a specific cancer, which comprises administering to a patient in need of such treatment, management or prevention a therapeutically or prophylactically effective amount of a selective tousled-like kinase inhibitory drug, or a pharmaceutically acceptable salt, solvate, or stereoisomer thereof, and a therapeutically or prophylactically effective amount of a second active ingredient, radiation therapy, hormonal therapy, biological therapy or immunotherapy.

3. A method of treating, managing or preventing a disease associated with undesired angiogenesis, which comprises administering to a patient in need of such treatment, management or prevention a therapeutically or prophylactically effective amount of a selective tousled-like kinase inhibitory drug, or a pharmaceutically acceptable salt, solvate, or stereoisomer thereof.

4. A method of treating, managing or preventing a disease associated with undesired angiogenesis, which comprises administering to a patient in need of such treatment, management or prevention a therapeutically or prophylactically effective amount of a selective tousled-like kinase inhibitory drug, or a pharmaceutically acceptable salt, solvate, or stereoisomer thereof, and a therapeutically or prophylactically effective amount of a second active ingredient.

5. The method of claim 1, wherein the cancer is advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, anaplastic astrocytoma, anaplastic oligodendroglioma, neuroendocrine tumor, rectal adenocarcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma, localized melanoma, malignant mesothelioma, malignant pleural effusion mesothelioma syndrome, peritoneal carcinoma, papillary serous carcinoma, gynecologic sarcoma, soft tissue sarcoma, scelroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, multiple myeloma, smoldering myeloma, indolent myeloma, fallopian tube cancer, androgen independent prostate cancer, androgen dependent stage 1V non-metastatic prostate cancer, hormone-insensitive prostate cancer, chemotherapy-insensitive prostate cancer, papillary thyroid carcinoma, follicular thyroid carcinoma, medullary thyroid carcinoma, or leiomyoma.

6. The method of claim 2, wherein the cancer is advanced malignancy, amyloidosis, locally advanced bladder cancer, metastatic transitional cell bladder cancer, relapsed brain tumor, progressive brain tumor, neuroblastoma, meningioma, hemangiopericytoma, multiple brain metastase, glioblastoma multiforms, glioblastoma, brain stem glioma, poor prognosis malignant brain tumor, malignant glioma, anaplastic astrocytoma, anaplastic oligodendroglioma, metastatic breast cancer, neuroendocrine tumor, rectal adenocarcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma, localized melanoma, malignant mesothelioma, stage MB non-small cell lung cancer, malignant pleural effusion mesothelioma syndrome, mutiple myeloma, peritoneal carcinoma, papillary serous carcinoma, gynecologic sarcoma, soft tissue sarcoma, scelroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage 1V non-metastatic prostate cancer, hormone-insensitive prostate cancer, chemotherapy-insensitive prostate cancer, papillary thyroid carcinoma, follicular thyroid carcinoma, medullary thyroid carcinoma, or leiomyoma.

7. The method of claim 3 or 4, wherein the disease or disorder is endometriosis, Crohn's disease, heart failure, advanced heart failure, renal impairment, diabetic retinopathy, retinopathy of prematurity, corneal graft rejection, neovascular glaucoma, retrolental fibroplasia, proliferative vitreoretinopathy, trachoma, myopia, optic pits, epidemic keratoconjunctivitis, atopic keratitis, superior limbic keratitis, pterygium keratitis sicca, sjogrens, acne rosacea, phylectenulosis, syphilis, lipid degeneration, bacterial ulcer, fungal ulcer, Herpes simplex infection, Herpes zoster infection, protozoan infection, Kaposi sarcoma, Mooren ulcer, Terrien's marginal degeneration, mariginal keratolysis, rheumatoid arthritis, systemic lupus, polyarteritis, trauma, Wegeners sarcoidosis, Scleritis, Steven's Johnson disease, periphigoid radial keratotomy, sickle cell anemia, sarcoid, pseudoxanthoma elasticum, Pagets disease, vein occlusion, artery occlusion, carotid obstructive disease, chronic uveitis, chronic vitritis, Lyme's disease, Eales disease, Bechet's disease, retinitis, choroiditis, presumed ocular histoplasmosis, Bests disease, Stargarts disease, pars planitis, chronic retinal detachment, hyperviscosity syndromes, toxoplasmosis, sclerosing cholangitis, rubeosis, endotoxemia, toxic shock syndrome, osteoarthritis, retrovirus replication, wasting, meningitis, silica-induced fibrosis, asbestos-induced fibrosis, veterinary disorder, malignancy-associated hypercalcemia, stroke, circulatory shock, periodontitis, gingivitis, macrocytic anemia, refractory anemia, or 5q-syndrome.

8. The method of claim 2 or 4, wherein the second active ingredient is anti-CD40 monoclonal antibody, histone deacetylyase inhibitor, heat-shock protein-90 inhibitor, insulin-like growth factor-1 receptor kinase inhibitor, vascular endothelial growth factor receptor kinase inhibitor, inducer of apoptosis in multiple myeloma cell, statin, insulin growth factor receptor inhibitor, lysophosphatidic acid acyltransrerase inhibitor, IkB kinase inhibitor, p38MAPK inhibitor, EGFR inhibitor, HER-2 antibody, VEGFR antibody, VEGFR inhibitor, P13K inhibitor, C-Met inhibitor, monoclonal antibody, anti-TNF-.alpha. antibody, hematopoietic growth factor, tousled-like kinase, anti-cancer agent, antibiotic, cox-2 inhibitor, immunomodulatory agent, immunosuppressive agent, corticosteroid, or a pharmacologically active mutant or derivative thereof, or a combination thereof.

9. The method of claim 7, wherein the second active ingredient is 2-methoxyestradiol, telomestatin, gefitinib, erlotinib HCL, trastuzumab, pertuzumab, bevacizumab, wortmannin, rituximab, tositumomab, edrecolomab, semaxanib, cyclosporin, etanercept, doxycycline, bortezomib, oblimersen, melphalan, G-CSF, GM-CSF, EPO, topotecan, pentoxifylline, taxotere, irinotecan, a COX-2 inhibitor, ciprofloxacin, dexamethasone, doxorubicin, vincristine, IL 2, IFN, dacarbazine, Ara-C, vinorelbine, isotretinoin, or a pharmaceutically acceptable salt, solvate, or stereoisomer thereof, or a pharmacologically active mutant or derivative thereof, or a combination thereof.

10. The method of any one of claims 1-4, wherein the selective tousled-like kinase inhibitory drug is an inhibitor of TLK1.

11. The method of any one of claims 1-4, wherein the selective tousled-like kinase inhibitory drug is an inhibitor of TLK1B.

12. A method for the treatment of diseases involving cell proliferation, migration or apoptosis of myeloma cells, or angiogenesis, which method comprises simultaneous, separate or sequential co-administration of effective amounts of: (i) an inhibitor of TLK or optionally in form of its tautomers, racemates, enantiomers, diastereomers and the mixtures thereof and optionally in form of the pharmacologically acceptable acid addition salts, solvates, hydrates, polymorphs, physiologically functional derivatives or prodrugs thereof; and (ii) at least a further chemotherapeutic or naturally occurring, semi-synthetic or synthetic therapeutic agent; in the form of a combined preparation, optionally adapted for a co-treatment with radiotherapy or radio-immunotherapy, to a person in need of such treatment.

13. A method for the treatment of diseases involving cell proliferation, migration or apoptosis of myeloma cells, or angiogenesis, which method comprises a simultaneous, separate or sequential co-treatment with an effective amount of an antagonist of at least one receptor selected from TLK1 and TLK2 or with a polymorph, metabolite or pharmaceutically acceptable salt thereof, and with radiotherapy or radio-immunotherapy.

14. The method of any one of claims 1-4, wherein the selective tousled-like kinase inhibitory drug is an antagonist of at least one kinase tousled-like kinase.

15. The method of any one of claims 1-4, wherein the selective tousled-like kinase inhibitory drug is used in combination with the adjuvant doxorubicin.

16. The method of any one of claims 1-4, wherein the selective tousled-like kinase inhibitory drug is a phenothiazine that inhibits TLK sensitized cell killing with RMT.

17. The method of any one of claims 1-4, wherein the selective tousled-like kinase inhibitory drug is selected from specific compounds perphenazine, promazine, promazine hydrochloride, thiethylperazine, thiorodazine, trifluoperazine and pharmaceutically acceptable salts, solvates, prodrugs, metabolites, polymorphs, tautomers, racemates, enantiomers, diastereoisomers or derivatives thereof.

18. The method of claim 17, wherein the selective tousled-like kinase inhibitory drug is enantiomerically pure.

19. A method of reducing or avoiding an adverse effect associated with radiation therapy, hormonal therapy, biological therapy, or immunotherapy in a patient suffering from a specific cancer, which comprises administering to the patient in need thereof a therapeutically or prophylactically effective amount of a selective tousled-like kinase inhibitory drug, or a pharmaceutically acceptable salt, solvate, or stereoisomer thereof.

20. The method according to any one of claims 1-4, wherein the selective tousled-like kinase inhibitory drug is administered in an amount of from about 1 to about 10,000 mg per day.

21. The method according to claim 2, wherein the selective tousled-like kinase inhibitory drug, or a pharmaceutically acceptable salt, solvate, or stereoisomer thereof is administered prior to, during, or after the administration of the second active ingredient, radiation therapy, hormonal therapy, biological therapy or immunotherapy.

22. A pharmaceutical composition comprising a selective tousled-like kinase inhibitory drug, or a pharmaceutically acceptable salt, solvate, or stereoisomer thereof, and a second active ingredient.

23. The pharmaceutical composition of claim 22, wherein the second active ingredient is anti-CD40 monoclonal antibody, histone deacetylyase inhibitor, heat-shock protein-90 inhibitor, insulin-like growth factor-1 receptor kinase inhibitor, vascular endothelial growth factor receptor kinase inhibitor, inducer of apoptosis in multiple myeloma cell, statin, insulin growth factor receptor inhibitor, lysophosphatidic acid acyltransrerase inhibitor, 1 kB kinase inhibitor, p38MAPK inhibitor, EGFR inhibitor, HER-2 antibody, VEGFR antibody, VEGFR inhibitor, P13K inhibitor, C-Met inhibitor, monoclonal antibody, anti-TNF-.alpha. antibody, hematopoietic growth factor, tousled-like kinase, anti-cancer agent, antibiotic, cox-2 inhibitor, immunomodulatory agent, immunosuppressive agent, corticosteroid, or a pharmacologically active mutant or derivative thereof.

24. The pharmaceutical composition of claim 23, wherein the second active ingredient is 2-methoxyestradiol, telomestatin, gefitinib, erlotinib HCL, trastuzumab, pertuzumab, bevacizumab, wortmannin, rituximab, tositumomab, edrecolomab, semaxanib, cyclosporin, etanercept, doxycycline, bortezomib, oblimersen, melphalan, G-CSF, GM-CSF, EPO, a cox-2 inhibitor, topotecan, pentoxifylline, ciprofloxacin, taxotere, irinotecan, dexamethasone, doxorubicin, vincristine, IL 2, IFN, dacarbazine, Ara-C, vinorelbine, isotretinoin, or a pharmaceutically acceptable salt, solvate, or stereoisomer thereof, or a pharmacologically active mutant or derivative thereof.

25. A method of treating a subject exposed to irradiation comprising the step of administering to the subject an effective amount of a tousled-like kinase activator, wherein the tousled-like kinase activator decreases morbidity and/or mortality of the subject exposed to irradiation.

26. The method of claim 25 when the tousled-like kinase activator is administered prior to exposure to irradiation.

27. The method of claim 25 when the tousled-like kinase activator is administered after the exposure to irradiation.

28. The method of claim 25, wherein the tousled-like kinase activator is dispersed in a pharmaceutically acceptable carrier.

29. The method of claim 25, wherein the amount of the tousled-like kinase activator that is administered is about 0.01 to 2.0 g/kg per day.

30. The method of claim 25, wherein the tousled-like kinase activator comprises one or more phenothiazine antipsychotic.

31. The method of claim 25, wherein the one or more phenothiazine antipsychotic is selected from the group comprising of chlorpromazine, fluphenazine, metaraminol and prochlorperazine.

32. The method of claim 25, wherein the irradiation is selected from 235U, 131I, 123I, 99Tc, 201Th, 133Xe, 125I, 60Co, and 137Cs, 60Co, 137Cs, 192Ir, 32P, 90Sr, 226Ra and a combination thereof.

33. A method of decreasing mortality of a subject that received an absorbed dose of ionizing radiation comprising the step of administering to the subject an effective amount of a tousled-like kinase activator to attenuate the effect of the absorbed dose.

34. A method of attenuating the damaging effects of an absorbed dose of irradiation in a subject comprising the step of administering to the subject an effective amount of a tousled-like kinase activator to attenuate the damaging effect of the absorbed dose.

35. The method of claim 34, wherein attenuating the damage results in a decrease in morbidity of the subjects.

36. A method of attenuating the damaging effects of an absorbed dose of irradiation in a subject comprising the step of orally administering to the subject an effective amount of a tousled-like kinase activator in combination with a radioprotective agent to attenuate the damaging effect of the absorbed dose.

37. A method of enhancing DNA repair in the tissues in a subject that received an absorbed dose of ionizing radiation resulting in radiation dermatitis comprising the step of administering an effective amount of a tousled-like kinase activator.

38. The method of claim 37, wherein the tissue is salivary gland tissue.
